(19) United States
(12) Patent Application Publication
(10) Pub. No.: US 2021/0190772 A1
(43) Pub. Date: Jun. 24, 2021

(54) ELECTRICALLY-MODULATED SURFACE WAVES AND AN ELECTRODE INTERFACE COMPRISING A METALLIC BILAYER

(71) Applicant: UNIVERSITY OF LOUISVILLE RESEARCH FOUNDATION, INC., Louisville, KY (US)

(72) Inventors: Sergio Brito Mendes, Louisville, KY (US); Martin O'Toole, Louisville, KY (US)

(21) Appl. No.: 17/174,658

(22) Filed: Feb. 12, 2021

Related U.S. Application Data
(63) Continuation-in-part of application No. 16/479,386, filed on Jul. 19, 2019.
(60) Provisional application No. 62/449,250, filed on Jan. 23, 2017.

(51) Int. Cl. G01N 33/543; G01N 27/30; G01N 27/327; G01N 33/569; G01N 21/64; G01N 21/17

(52) U.S. Cl. CPC G01N 33/5438; G01N 27/305; G01N 27/3277; G01N 2021/1721; G01N 21/648; G01N 21/1717; G01N 33/56983

(57) ABSTRACT

Immunosensors according to present embodiments combine a sandwich bioassay with an electrochemical surface plasmon resonance device for electrochemical detection of analytes from a sample, whereby a coated substrate for receiving an electroactive probe may be located in a flow cell, and the coated substrate comprises a first layer which is a silver (Ag) layer and a second layer which is a gold (Au) layer arranged so that the gold layer isolates the silver layer from an operating environment.

### CROSS-REFERENCE TO RELATED APPLICATION

This application is a continuation-in-part application of, and claims the benefit and priority to, U.S. Nonprovisional application Ser. No. 16/479,386, filed on Jul. 19, 2019, which claims the benefit of and priority to PCT Patent Application No. PCT/US18/14839, filed on Jan. 23, 2018, which claims the benefit of and priority to U.S. Provisional Application No. 62/449,250, filed on Jan. 23, 2017. The teachings and entire disclosure of all aforementioned applications are fully incorporated herein by reference.

### STATEMENT OF GOVERNMENT INTEREST

The invention described and claimed herein was made with government support under Grant No. 0814194 awarded by the National Science Foundation. The government has certain rights in this invention

### FIELD OF INVENTION

Embodiments described herein relate to molecular recognition through binding of antibodies or other molecules, labeled with spectroelectrochemically active complexes, to biomarkers followed by spectroelectrochemical analysis, and further embodiments relate to metallic bilayer structures for enhancing the sensitivity of electrochemical surface plasmonic wave devices, the practical applications of which include but are not limited to a methodology for direct detection and quantification of illnesses such as but not limited to infection diseases caused by an influenza virus, including without limitation the H5N1 influenza virus.

### BACKGROUND

Biological and chemical sensors are used increasingly in a wide variety of applications. Although such applications are numerous, some examples include the use of biological and chemical sensors as immunosensors, virus detection and identification, biochemical detection and characterization, metabolic profiling, testing for DNA-drug interactions, and food quality analysis. In some of these areas, the end result is limited by the amount of time required to obtain results. For example, some approaches work by detecting antibodies produced by a host's immune system in response to an infection, but current uses generally require that an infected individual has been infected for several days before any detection can be made.

The detection and identification of influenza strains is an area where quicker and more sensitive approaches are needed. Because current antiviral medications are most efficacious when administered in the first 48 hours of infection, the ability to detect and characterize influenza infection early in the infection process would significantly improve the ability of primary care physicians to effectively administer treatment. Earlier detection and characterization. e.g., within that 48 hour window, would also enable epidemiologists to defend more effectively against large-scale flu epidemics.

One example where enhanced sensitivity and speed are needed is with the detection of influenza in a patient. Current immunodetection techniques can distinguish between specific influenza viruses. For example, type A and type B influenza (and related subtypes) produce two main antigens: hemagglutinin (HA) and viral neuraminidase (NA), respectively. Each of these antigens is found on the exterior portion of the virus surface and binds to a specific antibody, making each type amenable to antibody trapping assays. Generally, some immunoassays involve an antibody for the target antigen being adsorbed to a solid phase, with the antibody-coated surface being exposed to a sample of blood or other biological fluid that may contain viruses or other pathogens the surface of which contains the target antigen. After an incubation period, a multistep washing is done to remove unbound antigen, and a second antibody with a label or a tag (referred to herein as a reporter antibody) is then added to enable detection of the antigen, or or other pre-determined analyte as selected. However, despite their capabilities with detection of proteins and small molecules in various fields, conventional immunoassays also present several disadvantages.

For example, while radioimmunoassays are generally sensitive and precise for determining concentration of analytes in a small sample size, they present an undesirable health hazard involved with usage of radioactive substances. And while enzyme immunoassays offer a relatively inexpensive means for detection, some specimens which undergo testing naturally contain inhibitors of the needed enzymatic activity. Chemiluminescence immunoassays offer some advantages in terms of sensitivity compared to enzyme immunoassays and radioimmunoassays, and the reagents used are relatively stable and relatively nontoxic. However, chemiluminescence immunoassays also require a high degree of precision by the technician related to the injection of hydrogen peroxide and the quenching of the light emission. Consequently, this type of immunodetection is more prone to false positives and operator error. Fluorescent immunoassays present good potential for high sensitivity and adaptability to various needs, but results are harder to interpret due to the production of autofluorescence from different organic substances normally present in serum.

Other approaches also present disadvantages that must be overcome. PCR-based approaches for viral detection (nucleic acid testing, being one example) provide reasonable sensitivity for identification and detection, but access to such testing is limited due to being predominantly performed at centralized laboratories, and requiring robust training of personnel to implement. Also, tedious and lengthy sample preparation is required, and based on current technologies the need for copying due to the concentration of pathogen DNA for detectable limits requires a long detection time (on the order of 2 hr. in many cases). The antigen-capture enzyme-linked immunosorbent assay (ELISA) has been explored to distinguish subtypes of influenza viruses, at times showing better sensitivity than immunoassays. Even so, ELISA is time consuming and takes prolonged times to provide results.

In yet another approach, potential-modulated attenuated total reflectance (PM-ATR) measures optical response (amplitude and phase delay) against several modulation frequencies for optical reconstruction of key features of the electrochemical process, which may include the faradaic current and the associated electron-transfer rate. However, this approach lacks sensitivity to probe redox species with weakly absorbing chromophores and/or at low surface concentrations. One reason is the surface density of analyte on an electrode is often low (less than 10⁻¹² moles/cm²) and the faradaic current which is generated—the signal truly linked to the species of interest—is consistently a small fraction of the total electric current, which also includes non-faradaic components. Similarly, sensors that utilize electrochemical approaches based solely on interpreting electrical signals, as done with voltammetric, amperometric, and impedimetric approaches, are limited by the inability to avoid non-faradaic components that usually hinder the interpretation of the electrochemical signal and faradaic processes produced by electron transfer.

Accordingly, a biosensor (or array of sensors) providing suitable sensitivity, specificity, and efficiency is needed for rapid detection and identification of biomarkers of diseases or other conditions, including virus antigens, pathogens, and other analytes. Present embodiments and alternatives provide this through a combined use of spectroelectrochemical techniques and optical detection of antigen recognition events.

### SUMMARY OF EMBODIMENTS

In some embodiments, novel optical impedance spectroscopy techniques incorporate a highly sensitive immunoassay-type approach with potential-modulated electro-active waveguide optical detection of antigen recognition events. Generally, the biosensor platforms are based on electro-active, integrated optical waveguides (EA-IOW), providing heightened sensitivity and specificity for detection. The biosensors employ spectroelectrochemical principles and methods, whereby surface functionalization of the EA-IOW with a capture antibody aimed at a specific virus antigen. The target antigen binds to (i.e., forms a chemical bond with) a capture antibody arranged within the device, thus promoting the chemical binding of a secondary antibody, i.e., a reporter antibody, labeled with an optical probe. In some embodiments, the optical probe is a methylene blue molecule that displays reversible changes in optical absorption throughout a reduction-oxidation transition.

The resulting optical signal is then electrically modulated and interrogated with high sensitivity by a propagating mode in the EA-IOW, in order to detect electron-transfer processes attendant with redox adsorbates. The term “redox events” refers to atoms or molecules having their oxidation state changed as electrons are transferred during a chemical reaction. Correspondingly, redox events are tracked by spectroscopically monitoring the absorbance/luminescence changes of a redox couple—i.e., an electro-active species conjugated with a reporter antibody as discussed herein—as electrical potential is modulated on an EA-IOW platform.

In some embodiments, optical signals are tuned spectrally to probe and reconstruct an electrochemical response to a specific faradaic process happening during reduction and oxidation of species. In doing so, the current embodiments make possible the development of diagnostic tests for pneumonia and influenza and their underlying causative agent with increased sensitivity, easily obtained specimens, and rapid turn-around for point-of-care deployment.

In the following descriptions of multiple embodiments, it will be appreciated that the ability to set up the structure of the inventive EA-IOW platforms for a selected wavelength being coupled into the device enables optical responses to be differentiated more precisely than with other spectroelectrochemical impedance techniques. In this manner, spectroscopic monitoring of absorbance/luminescence changes associated with a redox couple reduces the challenges associated with non-faradaic effects seen with other approaches. Moreover, differentiating the optical response with respect to time allows the reconstruction of the faradaic response of a particular redox process, from which the surface density, formal potentials, competing reaction pathways, and rates of the critical charge transfer events can be obtained. Accordingly, the detection limits of the approaches disclosed herein enable the measurement of redox couples with only a small difference in their molar absorptivities, and they can accommodate molecular assemblies that produce very low surface densities, with correspondingly low numbers of redox adsorbates that will display spectroelectrochemical activity.

Particular embodiments provided here are useful for immunosensor-based strategies for direct detection of specific viral pathogens in a biological sample, for example blood or other biological fluids. As a non-limiting example, other biological fluids include a sample from an intraocular cavity of the eye (i.e., the aqueous and/or vitreous humor) where infection might reside. Accordingly, in the present embodiments sandwich bioassays are disclosed which are incorporated as part of highly sensitive, single-mode, electro-active, integrated optical waveguide platforms. Working under alternating current (AC) potential-modulation enables detection and full electrochemical characterization of a redox protein sub-monolayer. Further still, labels that are based on redox probes are also provided, which aid in the detection of specific influenza virus antigens or other biomarkers.

These approaches enable one to more fully characterize antibodies labeled with spectroelectrochemically active redox probes, providing immunoassays for the detection and quantifications of virus antigens associated with influenza, pneumonia and other infectious diseases. The spectroelectrochemical characterizations provide a better alternative than currently available technologies for the creation of fully integrated immunosensor or other biosensor devices for point-of-care applications. One example is through offering the ability to detect pathogens within the incubation period as opposed to more time-consuming serological approaches that work based on detecting a certain antibody in the blood that has been produced by an infected person. In general, the characterization available from the practice of the embodiments herein and their alternatives is an improvement compared to electro-reflectance or transmission impedance approaches that are currently used.

Further still, in some embodiments, the inventive EA-IOW devices are reusable, requiring only washings with mild reagents to reset the sensor surface. In some embodiments, a flowcell is mounted on top of ultra-thin films described herein to provide an aqueous solution in which the electrolytic processes occur. The flowcell has an entry port (not shown) for introducing a biological sample into the device, as well as a port for egress of the sample, and also provides an easily-recycled, modular aspect of the sensing surface which allows conditions of the devices such as pH to be tailored for detection of different kinds of antigens. The ability to easily change the solutions on the surface of the waveguide without disrupting the optical alignment of the remainder of the components is another benefit afforded by these embodiments. In the context of flu season, for example, such modularity allows discrimination between different virus types or subtypes, which might involve different conditions to optimize detection and quantification. Similarly, specific seasons of the calendar might be associated with different propensities of exposure to infectious viruses. At the times when one virus subtype may be more prevalent, it is advantageous to easily change the solution on the surface of the waveguide while preserving optical alignment of the device.

Although specific mention is made of immunosensors involving antigen-specific virus detection and identification, the teachings and embodiments set forth here are not limited to this area. Rather, the platform disclosed herein can be tailored to produce devices for analyzing electrochemical and spectroscopic characteristics of various molecular assemblies to aid in pathogen recognition and direct detection through the techniques detailed herein, for use in a variety of settings which include other forms of biochemical detection and characterization, analysis of biological cells and tissues, metabolic profiling, DNA testing, and food quality analysis, to name a few.

### BRIEF DESCRIPTION OF THE FIGURES

The drawings, schematics, figures, and descriptions herein are to be understood as illustrative of structures, features and aspects of the present embodiments and do not limit the scope of the embodiments. Likewise, the scope of the application is not limited to any precise arrangements, scales, or dimensions as shown in the drawings, nor as discussed in the textual descriptions.

FIG. 1A illustrates schematically an electro-active, integrated optical waveguide (EA-IOW) device providing a sandwich bioassay with additional system components for spectroelectrochemical detection of an analyte, according to multiple embodiments and alternatives.

FIG. 1B provides a schematic of a EA-IOW with grating couplers, which is suitable for deposition of a ultra-thin indium tin oxide (UT-ITO) film for waveguide transmission of optical signals, according to multiple embodiments and alternatives.

FIG. 1C schematically illustrates differences in out-losses of optical signals associated with the waveguide of FIG. 1B, with and without the UT-ITO film.

FIG. 2 illustrates an EA-IOW for spectroelectrochemical interrogation and analysis of an analyte, according to multiple embodiments and alternatives.

FIG. 3 depicts the formation of a Ruthenium-labeled reporter antibody, according to multiple embodiments and alternatives.

FIG. 4 illustrates schematically various system components used in connection with an EA-IOW, according to multiple embodiments and alternatives.

FIG. 5 graphically depicts a measured optical signal based on AC-modulated absorbance for wave-guided light at 550 nm propagating on a EA-IOW during interrogation of an electro-active redox Cytochrome c (Cyt-C) protein.

FIG. 6A provides an optically reconstructed faradaic current density at the EA-IOW electrode for Cyt-C graphed against DC bias potential.

FIG. 6B graphs UV absorbance measurements of molar absorptivity of Cyt-C in solution for both oxidized and reduced states.

FIG. 7 graphs the optical absorbance results at 610 nm based on cyclic voltammetry (CV) scans using a EA-IOW according to present embodiments, for samples with virus antigen and samples without virus antigen.

FIG. 8A shows the absorbance amplitude measured under AC potential modulation for an EA-IOW of the present embodiments.

FIG. 8B graphs values for faradaic current density determined from optical absorbance data in FIG. 8A, based on electron transfer activity of a methylene blue-reporter antibody redox probe bound to a EA-IOW of present embodiments.

FIG. 9A graphs Faradaic current density for different volume concentrations of a virus antigen under varying bias potential.

FIG. 9B graphs faradaic current density for each volumetric concentration of antigen in FIG. 9A for the determination of a limit of detection for an EA-IOW, according to multiple embodiments and alternatives.

FIG. 10 illustrates schematically an electrochemical surface plasmon resonance (EC-SPR) system, according to multiple embodiments and alternatives.

FIG. 11A graphs values for an optical constants examination of a gold layer in air, schematically illustrated in FIG. 11B, at various excitation wavelengths, according to multiple embodiments and alternatives.

FIG. 11B illustrates schematically a gold film, according to multiple embodiments and alternatives.

FIG. 12A graphs values for an optical constants examination of a metallic bilayer in a buffer, schematically illustrated in FIG. 12B, at various excitation wavelengths, according to multiple embodiments and alternatives.

FIG. 12B illustrates schematically a metallic bilayer in a buffer, according to multiple embodiments and alternatives.

FIG. 13 graphs values for an optical constants examination of a metallic bilayer in a buffer at various excitation wavelengths, according to multiple embodiments and alternatives.

FIG. 14 graphs the optical constants examination of a redox probe layer in a buffer, according to multiple embodiments and alternatives.

FIG. 15 graphs the optical signal difference against the effective refractive index inside a prism at various excitation wavelengths, according to multiple embodiments and alternatives.

FIG. 16 graphs a reflectance under a CV scan for a redox probe immobilized on a metallic bilayer at different values of nsin(θ), according to multiple embodiments and alternatives.

FIG. 17 graphs a reflectance under a CV scan for a redox probe immobilized on a metallic bilayer using a desirable nsin(θ) at various wavelengths, according to multiple embodiments and alternatives.

FIG. 18A graphs cyclic voltammograms of a redox probe immobilized on a metallic bilayer and FIG. 18B illustrates the redox peak area of the surface coverage of the redox probe from FIG. 18A, according to multiple embodiments and alternatives.

FIG. 19A graphs the optical constants examination of a redox probe in a buffer using a six-layer structure at 637 nm, according to multiple embodiments and alternatives.

FIG. 19B graphs the optical constants examination of a redox probe in a buffer using a six-layer structure at 660 nm, according to multiple embodiments and alternatives.

FIG. 19C graphs the optical constants examination of a redox probe in a buffer using a six-layer structure at 685 nm, according to multiple embodiments and alternatives.

FIG. 19D graphs the optical constants examination of a redox probe in a buffer using a six-layer structure at 785 nm, according to multiple embodiments and alternatives.

FIG. 20A graphs the reflectance under a CV scan for a redox probe on a metallic bilayer at nsin(θ) 1.40 and a wavelength of 637 nm, according to multiple embodiments and alternatives. FIG. 20B graphs the reflectance under a CV scan for a redox probe on a metallic bilayer at nsin(θ) 1.42 and a wavelength of 637 nm, according to multiple embodiments and alternatives. FIG. 20C graphs the reflectance under a CV scan for a redox probe on a metallic bilayer at nsin(θ) 1.41 and a wavelength of 637 nm, according to multiple embodiments and alternatives.

FIG. 21A graphs the reflectance under a CV scan for a redox probe on a metallic bilayer at nsin(θ) 1.396 and a wavelength of 660 nm, according to multiple embodiments and alternatives. FIG. 21B graphs the reflectance under a CV scan for a redox probe on a metallic bilayer at nsin(θ) 1.409 and a wavelength of 660 nm, according to multiple embodiments and alternatives. FIG. 21C graphs the reflectance under a CV scan for a redox probe on a metallic bilayer at nsin(θ) 1.403 and a wavelength of 660 nm, according to multiple embodiments and alternatives.

FIG. 22A graphs the reflectance under a CV scan for a redox probe on a metallic bilayer at nsin(θ) 1.39 and a wavelength of 685 nm, according to multiple embodiments and alternatives. FIG. 22B graphs the reflectance under a CV scan for a redox probe on a metallic bilayer at nsin(θ) 1.402 and a wavelength of 685 nm, according to multiple embodiments and alternatives. FIG. 22C graphs the reflectance under a CV scan for a redox probe on a metallic bilayer at nsin(θ) 1.3953 and a wavelength of 685 nm, according to multiple embodiments and alternatives.

FIG. 23A graphs the reflectance under a CV scan for a redox probe on a metallic bilayer at nsin(θ) 1.374 and a wavelength of 785 nm, according to multiple embodiments and alternatives. FIG. 23B graphs the reflectance under a CV scan for a redox probe on a metallic bilayer at nsin(θ) 1.376 and a wavelength of 785 nm, according to multiple embodiments and alternatives. FIG. 23C graphs the reflectance under a CV scan for a redox probe on a metallic bilayer at nsin(θ) 1.376 and a wavelength of 785 nm, according to multiple embodiments and alternatives.

### MULTIPLE EMBODIMENTS AND ALTERNATIVES

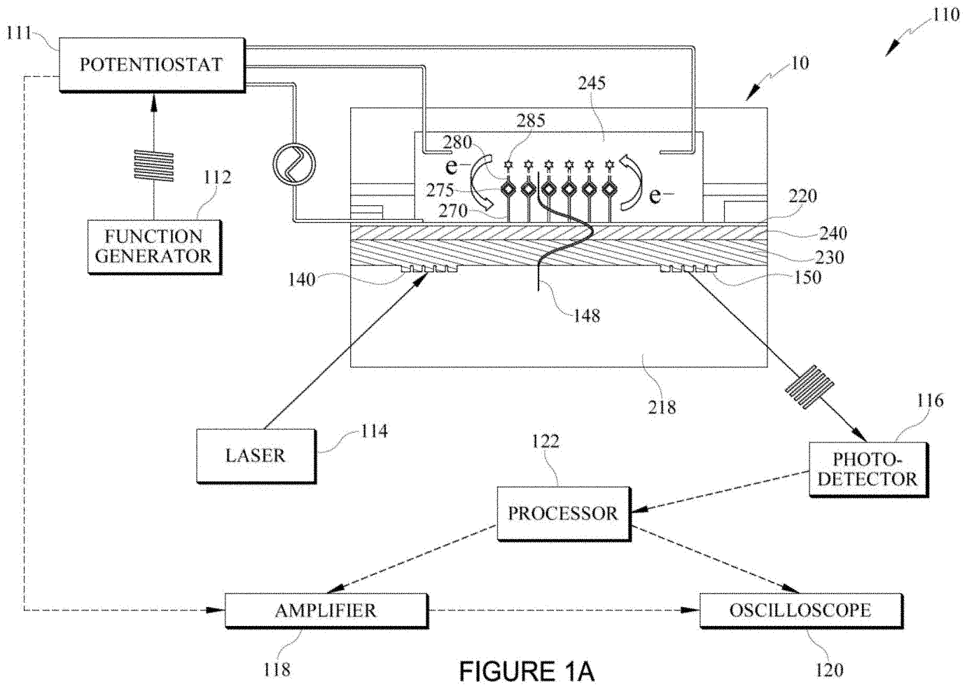

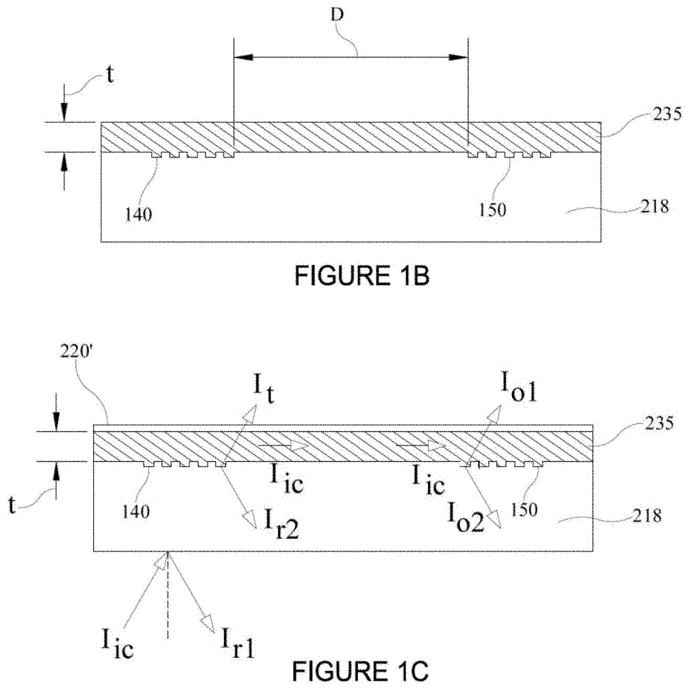

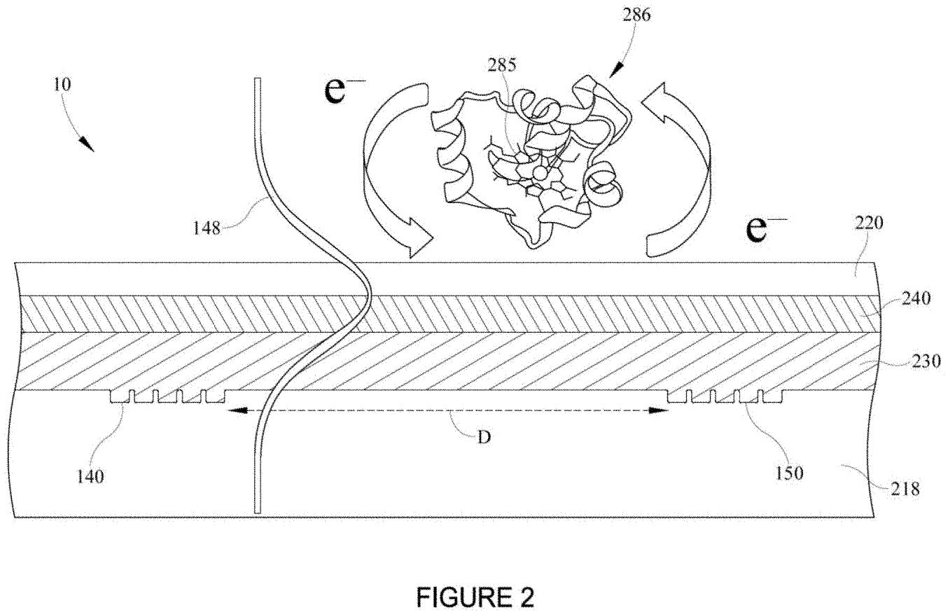

FIG. 1A generally illustrates an electro-active, integrated optical waveguide (EA-IOW) device 10 suitable for an immunoassay as part of a system 110 that includes other components described herein. The EA-IOW results in at least one primary antibody—sometimes referred to as capture antibody—bonded to at least one antigen; which becomes bound to a reporter antibody having a redox-active optical tag, i.e. probe. For ease of reference, in FIG. 1A only one each of the capture antibody 270, antigen 275, and reporter antibody 280 labeled with an electro-active species 285 to form a redox probe 286 as labeled and represented in FIG. 2.

The device 10 further comprises two transverse grating couplers shown as 140 and 150, the former permitting an optical beam (i.e., light beam) generated by laser 114 to enter the waveguide device 10, and the latter permitting egress of a modulated optical signal for further detection and analysis. Accordingly, the arrangement of the grating couplers 140, 150 causes the light beam to travel a path therebetween. In some embodiments, laser 114 is a linearly TE (transverse electric)-polarized laser beam aimed towards the edge of input grating coupler 140, operable at a wavelength of 610 nm or 633 nm). The grating couplers communicate with flowcell 245 so the optical beam generated by the laser interacts with a redox probe if one is present within the flowcell. The presence or absence of the redox probe depends on whether the analyte of interest, i.e., pre-determined analyte, is contained in the blood or other sample of biological fluid introduced into the flowcell.

Other components of system 110 illustrated schematically in FIG. 1A are a potentiostat 111 with a function generator 112 (e.g., DS345, Stanford Research Systems) connected to the potentiostat and providing a continuous wave (e.g., sinusoidal) creating and maintaining an electric field represented by 148 through a connection with a transparent electrode layer 220. Additional components illustrated in FIG. 1A include photodetector 116, signal amplifier 118, oscilloscope 120, along with a processor 122 for analyzing the detected signals.

As the absorbance or fluorescence signal of the probe is monitored under modulated potential, the selectivity and accuracy of the antibody-based recognition system are further enhanced by eliminating much of the effect of background absorbance or fluorescence associated with other biological components in the sample, and by the combination of redox potential and wavelength-controlled detection. In some embodiments, an absorbance or fluorescent probe has optical activity in the visible range of light. Alternatively, the response of the absorbing or fluorescent probes is in the near infrared (nIR) region, or other portions of the spectrum.

### Electro-Active Integrated Optical Waveguide (EA-IOW) Platform

In an embodiment, FIG. 2 is shown with similar structures as those identified for FIG. 1A. Accordingly, an EA-IOW 10 is shown having a semiconductor layer 220 forming a working electrode, which in some embodiments is indium tin oxide (ITO) film or graphene, that overcoats a highly transparent waveguide. In some embodiments, a transition layer separates the working electrode of the device from substrate 218. Optionally, such a transition layer can be formed from a combination of layers of silica (SiO₂) 240 and alumina (Al₂O₃) 230 films, or as desired the transition layer comprises a single layer such as alumina, in either case producing a multilayer stack. To persons of skill in the field of optical wave guides, methods such as atomic layer deposition (ALD) are known to provide desirable optical properties, whereby a thin film is grown through chemically covalent bonds. In such a method, a substrate 218 formed of glass or other transparent material is thoroughly cleaned in preparation for thin-film deposition, then coated with the thin film using an ALD vacuum deposition system (e.g., Savannah-100, Cambridge NanoTech Inc). By replicating the cycle for each deposition layer a certain number of times influenced by the desired thickness of the film, one can achieve a precise film thickness. Additionally, the self-limiting step of one atomic layer per cycle enhances the formation of a smooth film surface of uniform thickness over the substrate by allowing the growth of films that are conformal to the substrate surface.

### Fabrication of Ultra-Thin Indium Tin Oxide Films

This section describes a method of fabricating ultra-thin ITO films (UT-ITO films) suitable for a waveguide formed with grating couplers 140, 150 as described above, and which achieves excellent optical transparency, while retaining suitable electrical conductivity. The UT-ITO films, generally designated herein as 220′, are on the order of 30 nm or less, compared to more traditional ITO films in the 500 nm range, and these demonstrate high transparency and low resistivity for more precise extraction of optical data from the waveguide of the present embodiments. Initially, a substrate for the waveguide, such as a commercial glass substrate, is cleaned to prepare its surface. In an exemplary cleansing procedure, one or more substrates 218 is sonicated in an aqueous solution with 0.1% Triton X-100 soap (<3% polyethylene glycol) at 60° C., and both sides of the substrate are scrubbed with cotton swabs and rinsed with deionized water, a sufficient number of times to achieve removal of particulate or chemical impurities and to obtain a hydrophilic surface. Each substrate is then sonicated into Piranha Solution, acetone and methanol bath for 10 min at 60° C., with further rinsing between each step. Substrates are blown dried with nitrogen and heated in a 110° C. oven for 60 min, then removed from the oven to room temperature.

Following heating, an adhesion promoter such as hexamethyldisilazane (HMDS) layer is applied over one surface of each substrate, as each is spun at 3500 rpm for 30 sec as a means to improve the quality of a photoresist layer to be applied. For each surface treated with adhesion promoter, a solution of 1:1 of photoresist (Microdeposit, Shipley S1805) and J. T. Baker BTS-220 thinner are spin-coating onto the surface at 4000 rpm for 30 sec to form a film with thickness about 150 nm. Each substrate coated with photoresist is baked in an oven at 92° C. for 30 min to desired hardness. To prevent unwanted rear light scatter, the non-treated surface of each substrate (i.e., without photoresist layer) is dyed with an opaque coating such as black paint.

Each substrate is then placed in a suitable holder and its photoresist surface is exposed for a about 5 sec to a laser light with a wavelength of 442 nm (He—Cd laser line) adjusted at 4.4 mW·cm⁻² creating a shift in the interference pattern based on two-beam interference from Lloyd's mirror interferometer. Period length as a function of spatial frequency (i.e., the time for a complete wavelength cycle) is desirably controlled according to an incident angle following Equation (1),

$$Λ = \frac{λ}{2 n sin ( θ )} \qquad ( 1 )$$

where n, Λ, θ and λ are refractive index, pinch size, incident angle and incident wavelength respectively.

At this point, the opaque coating is removed from each substrate, and a solution with deionized water and Microdeposit Shipley 351 (4:1 ratio) is prepared. Each substrate is then placed in a suitable holder at Littrow configuration in relation to an aligned 632.8 nm laser beam. With monitoring of the diffraction beam with a light power meter detector and using data plotted with a LabView or other suitable program, the maximum intensity of the diffraction beam is detected, and each substrate is removed and submersed into deionizing water, then dried under a flow of nitrogen gas.

Each substrate is then prepared for engraving a series of sub-micron photo patterns on its treated surface, using reactive ion beam with etching. Preferably, the area between the patterns is protected using an adhesive such as Kapton tape, thereby defining a length representing a distance, D, between light input and output couplers, as shown in FIG. 1B.

After etching, the photoresist is removed from the appropriate surface of each substrate by standard cleaning procedures as described above, and the cleaned surface of each substrate is coated by Atomic Layer Deposition (ALD) with a transition layer 235 having a thickness less than 500 nm (e.g., 406 nm) in accordance with FIG. 1B. In some embodiments, transition layer 235 is a single layer, for example alumina; alternatively, the transition layer comprises sub-layers such as the ones illustrated for device 10 of FIGS. 1A and 2, i.e., silica (SiO₂) 240 and alumina (Al₂O₃) 230 films. At this point, each substrate represents a single-mode stack assembly waveguide ready for investigating optical properties of UT-ITO films on the order of 10 nm, which can be applied over a transition layer through known methods such as pulsed-DC reactive sputtering using a three-inch ITO target, 99.99% purity and 90% of In₂O₃ and 10% of SnO by weight.

In this sputtering technique, a sputter chamber is kept at 250° C. to evaporate water and other gases pumped into the chamber to maintain purity of the UT-ITO layers. A DC power is set at 230 W with the atmosphere inside the chamber controlled by Argon and oxygen flows to 5.5 mTorr at 100 sccm (standard cubic centimeter per minute) and 0.6 sccm, respectively. Deposition time for the UT-ITO is about 20 sec under spinning at 20 rpm for uniform coating of the UT-ITO film. After deposition, each stack is annealed using a hot plate at a temperature ramp increasing to 100° C. at 5 min to 200° C. at 15 min and leveling from 250° C.-275° C. from 20 min to 35 min. Simultaneously with this heating, UV radiation centered at 254 nm with irradiance 8 mW·cm⁻² is applied to balance the electrical and conductive properties of the UT-ITO film.

It is possible at this point to indirectly determine a thickness of each UT-ITO film, through envelope technique which can be accomplished on transparent substrates. In this technique, film thickness, h, is proportional to a difference between minimum transmittance values before and after the UT-ITO coating. The indirect measurement of film thickness is done according to Equation (2):

$$h = \frac{m Δ λ}{4 {n}_{ITO}} \qquad ( 2 )$$

where m is an integer associated with fringe order, Δλ is the difference between minimum transmittance values before coating and after, and n_{ITO} is the refractive index of ITO (1.88). In an exemplary calculation, m was 4 corresponding to forth order, and Δλ was approximately 35 nm producing a measurement of an 18 nm thick UT-ITO film. These data are factors in sheet resistance and extinction coefficient, as discussed below.

Sheet resistance can be monitored with a digital multimeter for measuring changes in conduction properties of a film layer of material brought about through annealing. At room temperature, oxygen vacancies (Vo) typically dominate the conduction mechanism in ITO films, such that conduction is through oxygen chemical bonds. Sheet resistance and resistivity of UT-ITO films, then, are measured before and after annealing. Table I shows the values of sheet resistance and resistivity before and after UV annealing, indicating an increase of resistance after thermal treatment.

TABLE I
Sheet resistance and Resistivity of UT-ITO films.

| | Sheet resistance (kΩ) | Resistivity mΩ · cm |
|---|---|---|
| Before annealing | 1.1 | 0.9 |
| After annealing | 1.8 | 1.4 |

The notable observation was UV thermal annealing as described above increased the resistivity of these UT-ITO films by around 36% ((1.4-0.9)/1.4). A probable explanation for this observation involves a rearrangement of chemical bonds by photons and thermal energy, thereby decreasing conduction centers Vo, and accordingly increasing sample resistivity.

In regard to further characterization of the UT-ITO films, Waveguide Mode Technique (WMT) can be used to compare waveguide transmission of optical signals with and without the UT-ITO film, in accordance with FIG. 1C. FIGS. 1B and 1C are shown with similar structures, the exception being FIG. 1C is viewed after the addition of UT-ITO 220′. With WMT, a single-mode 543 nm laser light is in-coupled by Bragg grating where an evanescence field interacts with a UT-ITO film. An incoming power signal, Intensity (I_{ic}), is measured based on intensity of reflection (Iᵣ₁+Iᵣ₂) and transmitted (Iₜ) signals. As an optical signal enters and passes along the waveguide path, a power meter measures the intensity of a reflection signal (Iᵣ₁+Iᵣ₂) from a light beam transmitted through grating (Iₜ) and that of its out-couple beam (Iₒ₁ and Iₒ₂). One then calculates intensity of the out-coupled signal based on the sum of out-coupling signal (Iₒ₁+Iₒ₂) as shown in FIG. 1C.

With the above data, Equation (3) is used to calculate signal out-losses (α) associated with the waveguide with and without the UT-ITO film.

$$α = - 10 log [ \frac{{I}_{o c}}{{I}_{i c}} ] = - 10 log [ \frac{{I}_{o 1} + {I}_{o 2}}{{I}_{i} - ( {I}_{r 1} + {I}_{r 2} + {I}_{t} )} ] \qquad ( 3 )$$

As previously alluded to, WMT was applied before and after the UT-ITO coating to compare light transmission losses with and without the UT-ITO film. In this way, signal transmission losses are calculated in view of the presence of the UT-ITO film (α′) based on losses from waveguide pre-film (αWG) and total losses with UT-ITO film (αUT-ITO). Having this difference enables a calculation of extinction coefficient (k) of associated with each UT-ITO film layer. Table II shows losses of waveguides with and without coating for eight samples.

TABLE II
Losses measured experimentally by WMT
from waveguide with and without UT-ITO.

| | Sample number | | | | | | | |
|---|---|---|---|---|---|---|---|---|
| | S1 | S2 | S3 | S4 | S5 | S6 | S7 | S8 |
| α_{WG} (dB/cm) | 3.4 | 4.1 | 2.5 | 2.9 | 4.3 | 5.7 | 6.7 | 4.1 |
| α_{UT-ITO}(dB/cm) | 7.4 | 7.1 | 6.1 | 7.3 | 7.5 | 8.9 | 8.1 | 9.6 |
| α′ (dB/cm) | 4.0 | 3.0 | 4.4 | 4.4 | 3.2 | 3.2 | 1.4 | 5.5 |

An extinction coefficient (k) of UT-ITO films is given by Equation (4), where α′ is loss of ITO, h is thickness of UT-ITO layer, λ is wavelength of laser beam and S is sensitivity factor of the aluminum waveguide. Determination of the sensitivity factor enables one to calculate k.

$$k = \frac{{α}^{′} λln ( 10 )}{40 π hS} \qquad ( 4 )$$

Sensitivity factors are an inherent characteristic of waveguide design, as are distance between couplers (D) and thickness (t), in view of fabrication materials, and refractive indices of the respective layers (substrate, transition layer, and electrode). By combining these features, one can determine effective values of thickness (t_{eff}) and refractive index (N_{eff}) to help induce and enhance electromagnetic wave propagation along the EA-IOW.

In view of such parameters, various equations are helpful for extracting effective thickness and effectual refractive indices oft. For purposes of the present disclosure, using a refractive index of alumina (n_{f}=1.645 at 543 nm) grown on a glass substrate (nₛ=1.509) and kept in air (n_{c}=1), Maxwell's equations enables one to define an effective refractive index of waveguides. In this regard, N_{eff} is a virtual value of all refractive indices combined that a waveguide mode will travel into.

In view of applying proper boundary conditions, N_{eff} is given by Equation (5),

$$\frac{2 µt}{λ} \sqrt{( {n}_{f}^{2} - {N}_{eff}^{2} )} = \\ {tan}^{- 1} [ \sqrt{( \frac{{N}_{eff}^{2} - {n}_{s}^{2}}{{n}_{f}^{2} - {N}_{eff}^{2}} )} ] + {tan}^{- 1} [ \sqrt{( \frac{{N}_{eff}^{2} - {n}_{c}^{2}}{{n}_{f}^{2} - {N}_{eff}^{2}} )} ] + {m}^{′} π \qquad ( 5 )$$

where λ is wavelength of propagating light, t is thickness of the transition layer, e.g., alumina, and m′ is guided mode (zero for present calculation). Because Equation (5) does not have a close solution for N_{eff}, a numerical calculation using a Newton-Raphson equation was used to find successively better approximations to the root of the real value for N_{eff}. Computerized calculations were then obtained in view of waveguide parameters described herein, to reach a finding of N_{eff} equal to 1.585, a figure consistent with overall data concerning the conductivity of the UT-ITO films.

Taking into account several values mentioned above, effective thickness (t_{eff}), is given by Equation (6), and is dependent on t, N_{eff}, n_{c} and nₛ:

$${t}_{eff} = t + \frac{λ}{2 π \sqrt{( {N}_{eff}^{2} - {n}_{c}^{2} )}} + \frac{λ}{2 π \sqrt{( {N}_{eff}^{2} - {n}_{s}^{2} )}} \qquad ( 6 )$$

After substitution of values, then, t_{eff} for for the waveguide is calculated to be approximately 654 nm, and from this the thickness of the UT-ITO film can be determined. Likewise, when a laser beam is used as described above to creating an interference pattern based on two-beam interference, it establishes a shift representing the difference in Transmittance between the substrate/alumina structure and the substrate/alumina/UT-ITO structure. The degree of shift as a function of wavelength can then be used as another basis for extracting the thickness of the UT-ITO film layer, which is on the order of less than 40 nm, with embodiments capable of reaching approximately 18 nm thick for this layer.

As alluded to previously, sensitivity factor is associated with the extinction coefficient, and can be determined by the following Equation (7).

$$S = \frac{2 {n}_{l} L ( {n}_{f}^{2} - {N}_{eff}^{2} )}{{N}_{eff} {t}_{eff} ( {n}_{f}^{2} - {n}_{c}^{2} )} \qquad ( 7 )$$

Using waveguide parameters described herein, sensitivity assumes the value of 14×10³ for all samples. With reference back to Equation (4), replacement of parameters for each sample enables the calculation of excitation coefficients. According now to Equation (8), transmittance, T, depends on exponential function of extinction coefficient (k) and the UT-ITO film's thickness, h.

$$T = 1 - {e}^{- ( \frac{4 π k h}{λ} )} \qquad ( 8 )$$

Accordingly, Table III summarizes the results from 8 sample waveguides characterized for extinction coefficient and transmittance in accordance with the analytical methods detailed above.

Sample number

| | S1 | S2 | S3 | S4 | S5 | S6 | S7 | S8 |
|---|---|---|---|---|---|---|---|---|
| k (×10⁻³) | 1.2 | 0.9 | 1.1 | 1.3 | 1.0 | 0.8 | 0.3 | 1.3 |
| T (×10⁻⁴) | 2.2 | 1.6 | 2.0 | 2.5 | 1.8 | 1.5 | 0.6 | 2.4 |

In view of a technique for fabricating UT-ITO films for use with the inventive waveguides, in some embodiments UT-ITO films obtained by pulsed-DC reactive sputtering provide a good balance between extinction coefficient and resistivity, which is useful for the electro-active optical waveguides of the present embodiments. At thicknesses outlined herein, ITO can be highly transparent and conductive at the same time, and at ultra-thin scalethe effective values of thickness (t_{eff}) and refractive index (N_{eff}) determined herein induce and enhance electromagnetic wave propagation.

### Functionalization of the EA-IOW Interface

To prepare an assay, a surface of the device 10 is functionalized with an additional monolayer. For example, in some embodiments functionalization is performed at the UT-ITO or ITO layer (referred to for the remainder of this disclosure as simply “ITO”), graphene layer, or other electrode surface layer as selected by a user. In some embodiments, the ITO electrode surface is coated with (3-Aminopropyl)triethoxysilane (APTES) to generate an amine-bearing surface. For example, the ITO surface of the EA-IOW is immersed into a solution containing 10 mL of 2-propanol, 100 μL of APTES, and 4 to 5 drops of deionized water, then incubated a sufficient period of time such as overnight. Following incubation, the device surface is thoroughly rinsed with 2-propanol and DI water, then dried under a nitrogen flow. In this way, the surface modification facilitates conjugation/adsorption at the site, serving as a foundation for joining successive protein monolayers to the device.

As desired, labeling the amine groups of APTES with molecules serving as fluorescent probes and imaging the single molecules with confocal fluorescent microscopy provides a means to determine the presence and reactivity of the APTES overlayer on the EA-IOW device. As desired, carboxylate-modified fluorosphere beads (F8787, Thermo Fisher Scientific), which have a typical size of about 20 nm and feature an absorption peak at 505 nm and a maximum fluorescence emission at 515 nm, may be bound to the surface amine sites for this purpose. Cresyl violet is a dye with an excitation wavelength of approximately 540 nm, and the fluorescence yield is proportional to concentration of the dye. Acidified forms of cresyl violet such as cresyl violet perchlorate binding to the protein-coated overlayer and permit themselves of fluorescent detection. These forms as well as similar dyes and stains as known in the art also will serve as suitable examples of fluorescent probes for detecting presence and reactivity at this layer.

Next, in some embodiments a protein monolayer (e.g., native or recombinant protein G, i.e., ProG) is adsorbed to the modified surface layer, which facilitates capturing and properly orienting the molecules of the capture antibody on the surface. Recombinant ProG is a genetically engineered form of the native protein with the serum albumin binding region removed so that albumin can be utilized as a blocking buffer. It is a conventional antibody-binding protein adaptable for a wide range of immunoassay systems, which in present contexts promotes the capture and suitable orientation of the antigen-binding sites in the immobilized capture antibodies. For example, it is desirable for those binding sites should be oriented away from the solid phase for optimum conditions to capture the target analytes. Alternatively, protein A (ProA) is a suitable protein for the monolayer to provide functionalization of the electrode.

ProG serves well in the functionalization of the inventive EA-IOW's for several reasons. Covalent conjugation of ProG to the APTES surface can be accomplished through known amide bond-forming chemistry. Further, ProG has two IgG binding sites, which reduces the chance that surface immobilization will block the binding sites. In addition, ProG binds IgG antibodies with very high affinities on the Fc region. Also, adsorption of ProG on the ITO/APTES surface produces acceptable electron-transfer rates through the ProG layer, and antigen binding will be enhanced through the adsorption of capture antibodies to the ITO/APTES/ProG surface. Accordingly, in an embodiment, the functionalizing the sensing surface of the EA-IOW proceeds in several stages, i.e.,

bare ITO,

then ITO/APTES,

then ITO/APTES/ProG,

then ITO/APTES/ProG/Capture Antibody.

As the ITO/APTES/ProG/Antibody layers are made ready for assaying, suitable blocking buffers (e.g., containing 1% Bovine Albumin Solution, BSA) are utilized to reduce non-specific adsorption interactions on the surface of the electrode.

In use during assaying, the stages then proceed as follows:

ITO/APTES/ProG/Capture Antibody/Antigen,

then ITO/APTES/ProG/Capture Antibody/Antigen/Reporter Antibody.

For the latter stage, in rounding out the sandwich bioassay, the reporter antibody will be labeled with an electro-active species serving as an optical probe, thereby providing a signature spectroelectrochemical response for detection as described herein.

To renew the sensing surface after use and achieve reusability, the ITO/APTES/ProG/Ab EA-IOW surface can be sonicated in a potassium carbonate solution at pH 9-11. This reverses the interaction between ProG and ProG/Ab with the ITO/APTES surface, without undue compromise of EA-IOW performance such as through coupling loss or increased resistance to spectral transmission.

While ProG and ProA have been described, alternative embodiments may also involve functionalization by adsorbing antibody directly to the surface 220 of the electrode or an additional layer bound to the electrode such as an APTES monolayer.

In use, once a protein monolayer is established over the ITO/APTES surface, the capture antibodies are introduced to the assay device. One or more antibodies should be introduced in an appropriate buffer system (e.g., phosphate buffered saline or 4-(2-hydroxyethyl)-1-piperazineethanesulfonic acid, i.e., HEPES). The antibodies are generated through methods which are known in the art, and include generating the antibodies in mammals such as mice, rabbits, or others. The pH and salt concentration of the solution should remain at appropriate levels to maintain the solubility of the antibody and so the antibody does not undergo denaturing. In an embodiment in which device 10 is used for immunoassaying of influenza, for example, capture antibodies are selected which bind to known antigens related to influenza viruses of interest, such as hemagglutinin (HA) or viral neuraminidase (NA), for example.
Likewise, various blocking buffers as known in the art to help in reducing background interference are within the scope of embodiments contained herein even if not expressly described. It will be appreciated that, depending on the types of viruses involved and the conditions under which their detection is to occur, the embodiments herein are to be considered broadly to include other capture antibodies (polyclonal or monoclonal), reporter antibodies, dyes, surface modifiers, and redox probes in addition to those described herein.

### Performance of the Functionalized Sensor Using a Redox Probe

In preparation for detection of analytes, reporter antibodies are selected which also specifically bind to the antigens (such as HA or NA antibodies in the present example). In some embodiments, depending on the antigen, the same antibody is used for both capture antibody and reporter antibody, for example, monoclonal anti-H5 (H5N1) antibody, as antibodies tend to be highly specific in which antigens they will bind to. Once the antigens, in this case HA, are captured by the capture antibody and immobilized on the device surface, it enables the binding of the antigen to the reporter antibody which is labeled with an electro-active species having optical properties, e.g., methylene blue. Further still, there may be some contexts—such as when the target is very large (e.g., a whole cell or a virus) as opposed to a single protein antigen—where enhanced sensitivity can be achieved by using capture and reporter antibodies that are different from each other, wherein the reporter antibody molecule binds to a second binding site on the target.

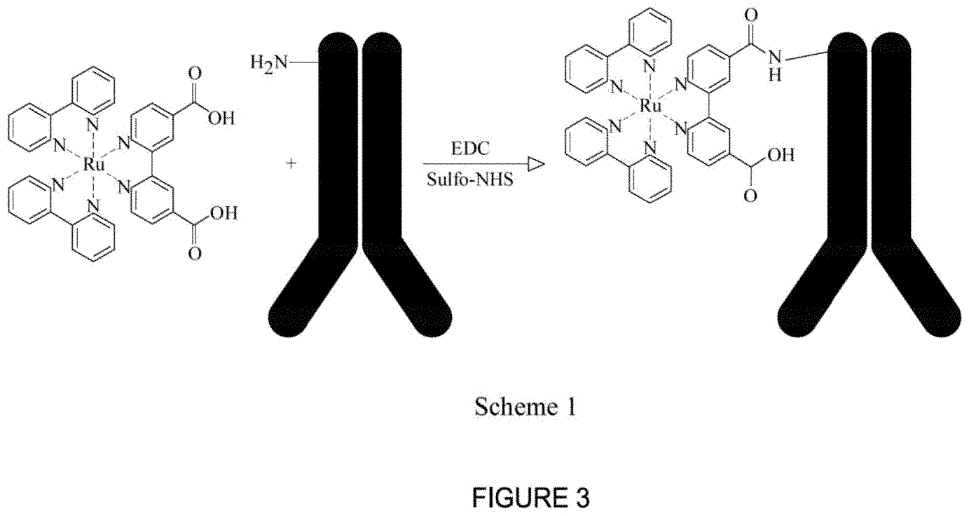

Before they are introduced to the assay, during synthesis the reporter antibodies (i.e., which can be multiple copies of a single type of antibody) are labeled with a suitable redox-molecule, i.e., electro-active, which acts as the probe whose optical properties can be measured. Among a number of possible candidates, a complex expected to have a favorable spectral response is bis(2,2′-bipyridine)(2,2′-bipyridine-4,4′-dicarboxylic acid)ruthenium(II). FIG. 3 represents the formation of a Ruthenium-labeled antibody. Bipyridine derivatives such as this display well-characterized absorbance and fluorescence behavior as a function of the Ruthenium oxidation state, with an optical signal of sufficient longevity for detection. Furthermore, these derivatives contain pendant carboxylic acid groups to facilitate the conjugation between the antigen and the reporter antibody as shown in FIG. 1. Typically, labeling occurs by mixing a reporter antibody for a sufficient period of time with methylene blue (or other redox-active molecules as known in the art that might be selected for the optical probe) until conjugation occurs, then as desired purifying using a standard resin column and known buffers. Methylene blue is an acceptable label because it shows a strong and reversible change in optical absorption between 600-660 nm while coupled to a transition metal in its oxidation state.

Suitable molar ratios for Ruthenium to the antibody as part of the labeled reporter antibody may fall within a range of about 1:1 to 10:1. It is expected that the lower end of this range will suffice for suitable antigen binding, particularly for antibodies with abundant lysine amino groups on the surface of the antibody, while the higher end of the range or even higher will provide greater signal amplification of antigen recognition events. Working in an aqueous solvent system, a RuAb complex according to these embodiments displays prominent redox potentials at −800 mV, −250 mV, and 250 mV.

In general, suitable redox probes will employ electro-active species that undergo a reversible redox process, which are accessible in aqueous solution and have a detectable change in optical properties tied to the redox event. Further, the electro-active species selected for the redox probe will conjugate with a protein selected as the aforementioned reporter antibody. In the above-mentioned bipyridine ruthenium complex, a number of transition metals would serve as acceptable substitutes for ruthenium, e.g., platinum, iron, and molybdenum. In addition to transition metals, other redox molecules (e.g. methylene blue) can be used for the redox probe.

Accordingly, there are various options and approaches for selecting a redox probe. A suitable redox probe is one that displays multiple accessible oxidation states, and is capable of binding to one or more organic compounds and to a selected reporter antibody. When bound in this fashion, the redox molecule acts as a probe based on its optical properties (absorbance and/or fluorescence spectra) which are dependent on the oxidation state of the redox probe, and otherwise displays a detectable change in optical properties when the redox molecule is oxidized/reduced. When a suitable voltage is applied to a substance or solution containing the redox probe, a spectral change in the absorbance or fluorescence associated with the probe will change as the oxidation state of the redox molecule changes. If the applied potential is modulated, then the optical signal for the probe is also modulated, so that both the signal and its modulated behavior can be detected.

In accordance with the factors mentioned herein, non-limiting examples of other compounds and derivatives for labeling the reporter antibodies with redox probes include methylene blue, Methylene blue succinymide, methylene blue maleimide, Atto MB2 maleimide (Sigma Aldrich) and other methylene blue derivatives; 3,7-Bis-[(2-Ammoniumethyl) (methyl)amino]phenothiazin-5-ium trifluoroacetate; 3,7-Bis-(piperazin-4-ium-1-yl)phenothiazin-5-ium trifluoroacetate; 3,7-Bis-[(2-ammoniumethyl)(methyl)amino]phenothiazin-5-ium chloride; and 3,7-Bis-(piperazin-4-ium-1-yl)phenothiazin-5-ium chloride, to name some.

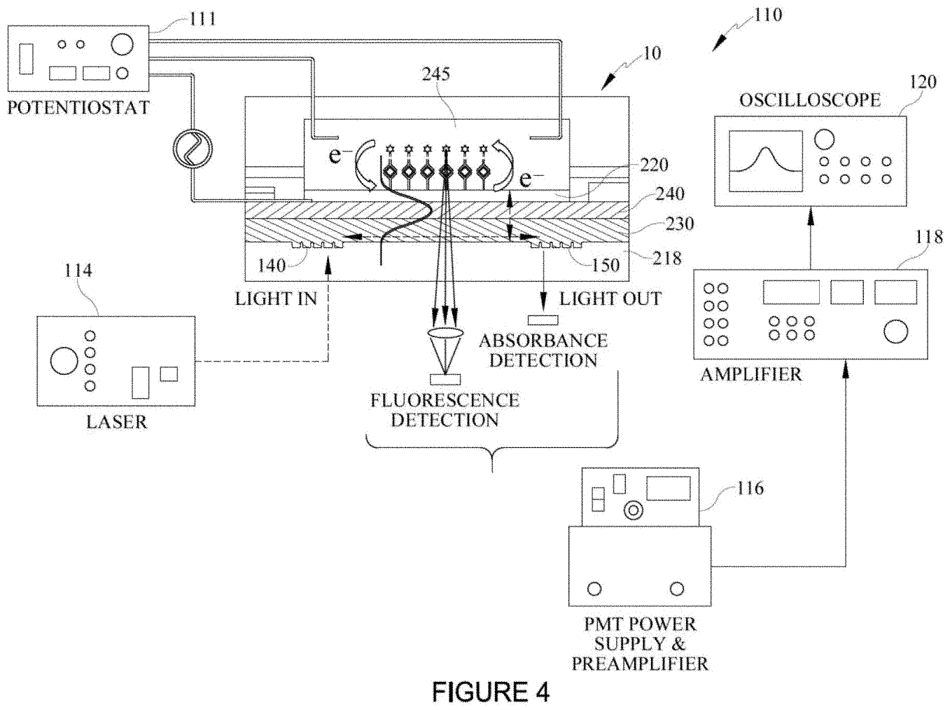

Turning now to FIG. 4, a schematic is provided of various components used in connection with device 10 to interrogate spectroscopic activity and characterization related to surface-adsorbed molecular layers. The modulated potential within the system is controlled by a standard potentiostat with inputs to the device. Other components of system 110 may include potentiostat 111, laser 114 (e.g., solid state, 550 nm) for generating the light beam that enters grating coupler 140, one or more photodetectors 116 (e.g., a photomultiplier detector) that receives the outcoupled optical signal from grating coupler 150, and optionally monochromator (not shown) and Imaging Charge Couple Device (“ICCD”) (not shown) as known in the art for obtaining spectrally-resolved measurements, as well as a suitable amplifier 118 to amplify the generated signal, and an oscilloscope 120 for data collection and representation along with various processors as described herein for controlling voltage inputs that maintain an electrical field and for interpreting optical and electrical signals.

To apply these techniques to the broadband spectroscopic characterization of a protein submonolayer, the beam from a laser source is routed via optical fiber to a grating coupler serving as a light beam entry port of the device 10. In some embodiments, a highly anamorphic optical beam from a laser source (not shown) is utilized with large divergence (high NA) in the plane of incidence, but fairly collimated in the lateral direction of the planar optical waveguide. As desired, diffraction-limited optical components are deployed to launch the light beam towards the integrated grating coupler 250. In this regard, within the scope of present embodiments, various components are suitable for enhancing the spectral bandwidth of a free-space propagating optical beam coupled into an EA-IOW at the input grating coupler in order to shape the light beam delivered to device 10 by a laser or other optical light beam source as shown in FIG. 4. As discussed further below, working with a highly anamorphic optical beam at the aplanatic condition for broadband coupling, a solid immersion cylindrical lens enhances the numerical aperture of laser light delivered to a grating coupler.

To couple a light beam propagating in free space into an optical mode of a planar waveguide using a grating coupler, the following well-known synchronous condition must be satisfied:

$${N}_{eff} = {n}_{0} {sin θ}_{0} + \frac{λ}{Λ} , \qquad ( 9 )$$

where N_{eff} is the effective refractive index of the waveguide, and λ is the wavelength of the light in vacuum. Further rounding out this equation, n₀ is the refractive index of air, θ₀ is the angle between the incident light beam and the normal to the waveguide surface designated by broken line perpendicular to the waveguide in FIG. 1C, and Λ is the period of the grating couplers. These are the two transverse grating couplers shown as 140 and 150, respectively in FIGS. 1A-C and FIG. 2, which are fully embedded into the laminar structure of the EA-IOW of present embodiments.

The central wavelength λ_{c} of the grating coupler, defined at θ₀=0 deg, is then given by

λ_{c}=ΛN_{eff}, (10)

and the spectral bandwidth provided by the grating coupler can be approximated by

Δλ≈2ΛNA, (11)

where NA is the numerical aperture of the incident optical beam. It will be appreciated the period of the grating coupler can be chosen in relation to a selected central wavelength coupled into a particular waveguide.

To enhance the working NA delivered to a grating coupler of the inventive EA-IOW, a solid immersion lens augments the numerical aperture of an incident light beam coupled into the EA-IOW, desirably keeping the amount of spherical aberration low. The basis for such an enhancement is that the spectral bandwidth is determined by the numerical aperture of the light beam striking the grating coupler, and the coupled bandwidth scales directly with the numerical aperture of the optical beam.

To achieve the desired effects, in some embodiments a collimated optical beam is bent towards the optical axis by a first cylindrical lens (not shown) and propagates as a converging cylindrical wave. This cylindrical wave then impinges on a second cylindrical lens, which is designated herein as a solid immersion lens and optionally is attached to the waveguide substrate itself. The cylindrical surface of the second cylindrical lens has two axial conjugate points, which are referred to as aplanatic points. A first aplanatic point is located at the grating coupler, and a second aplanatic point is located at the back focal point of the cylindrical lens. Some advantages are realized if the two cylindrical lenses and the waveguide substrate are made from the same glass (i.e., have the same refractive index). Suitable cylindrical lenses for these purposes are plano-convex lenses made of BK-7 glass according to specifications listed in Table IV.

TABLE IV
Lens selection for augmenting numerical aperture

| | Melies Griot | efl | bfl | Radius | Center thickness | |
|---|---|---|---|---|---|---|
| Lens | cat. no. | (mm) | (mm) | (mm) | (mm) | NA |
| 1 | 01LCP025 | 10.0 | 7.4 | 5.2 | 3.8 | 0.45 |
| 2 | 01LCP023 | 5.0 | 3.5 | 2.6 | 2.3 | 0.45 |

While other enhancements are attainable and adaptable for use in the present embodiments, the ability to obtain potential-modulated fluorescence measurements occurs as the guided optical beam propagates between structures 140 and 150 seen in FIG. 1A, and excites light emission (i.e., absorption) from surface-immobilized fluorophores (i.e., chromophores). With continued reference to FIG. 4, a set of micro-optical components (including notch and color filters) attached to the back of an exemplary EA-IOW platform collect the emitted radiation according to known methods. In potential-modulated absorbance measurements, the optical attenuation by the surface-adsorbed chromophores can be determined from the guided optical beam that is outcoupled by another grating coupler (not shown) and routed to an optical fiber using a similar set of anamorphic optical components. The collected optical signal is then coupled to a photodetector, or optionally an ICCD detector, for spectroscopic measurements. The signal which is detected is then electronically processed by a current pre-amplifier and sent to a lock-in-amplifier (e.g., SR810, Stanford Research Systems). A three-electrode potentiostat (CHI 660D) controls the electric potential applied to the device, and provides a trigger to the lock-in-amplifier for impedance measurements.

Accordingly, through monitoring of spectroelectrochemical transduction afforded by device 10, optical data is obtained and analyzed, the results of which are used to reconstruct the electrochemical information of faradaic processes associated with electron transfer and the ensuing changes in oxidation state of a redox species at the electrode, including a redox-labeled reporter antibody discussed above. The absorbance of light by the probe at a known wavelength enables one to determine, as a function of the optical measurements, the faradaic current density against potential,

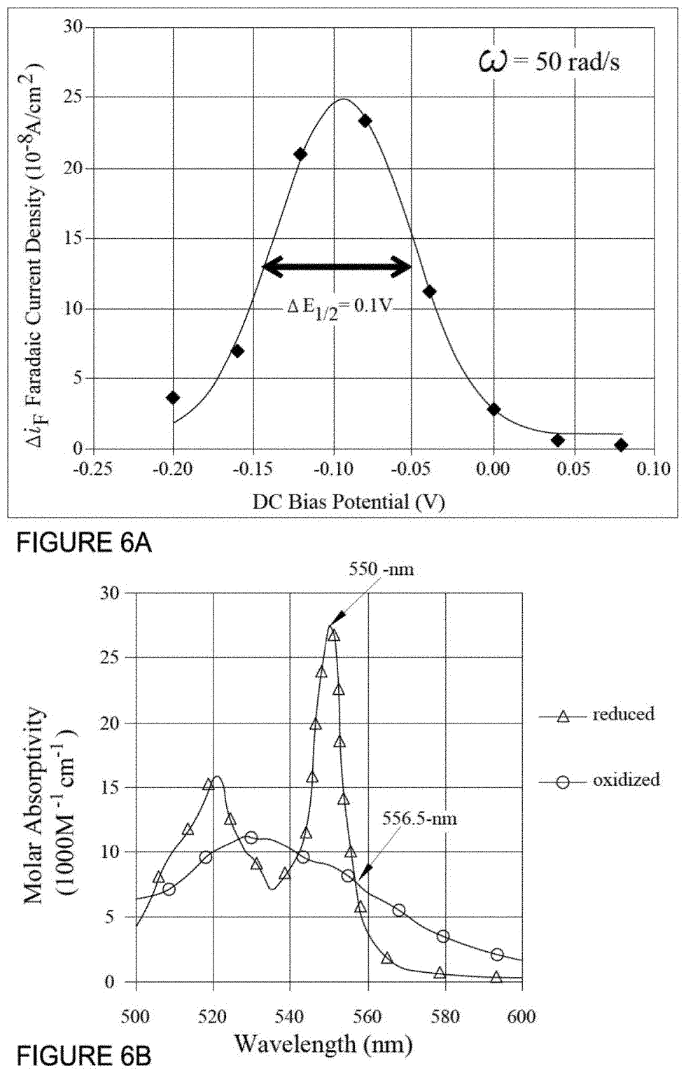

i_{F}=(nF)/(SΔ_{ϵ})*d_{A}/dₜ, (13)

where Δϵ is the molar absorptivity difference at the known wavelength between the two redox states; n is the number of electrons; and F is the Faraday constant, respectively. For suitable probes according to the embodiments herein, when faradaic current is plotted against potential, as shown in FIG. 6A. As appreciated in viewing FIG. 6B, comprising a plot of UV absorbance measurements against molar absorptivity of Cyt-C in solution at oxidized and reduced states, the cathodic and anodic peaks generally have similar magnitudes and are co-located at the formal potential of the redox process.

Through empirical data, mathematical relationships can be used to correlate optical data with electro-chemical information of redox events occurring at the device electrode. Under an AC potential modulation, E=E_{dc}+ΔE_{ac} sin(ωt), a modulated optical signal is measured for a light beam propagating along device 10 between points 140 and 150. Optical data, which is associated with the effects on the probe as the light propagates, is then obtained both in the absence of the redox adsorbate (i.e., baseline) according to Equation (13),

I₀=I_{(dc,0)}+ΔI_{(ac,0)} sin(ωt+θ_0), (13)

and in the presence of the redox adsorbate according to Equation (14),

I=I_{dc}+ΔI_{ac} sin(ωt+θ). (14)

In this fashion, the in-phase and out-of-phase components of the AC modulated optical absorbance can be determined for both phases, with use of Equations (15) and (16), respectively:

$$\begin{matrix}Δ {A}_{ac , in} = - \frac{Δ {I}_{a c} cos ( θ )}{{I}_{d c} ln ( 10 )} + \frac{Δ {I}_{a c , 0} cos ( {θ}_{0} )}{{I}_{d c , 0} ln ( 10 )} & ( 15 ) \\ Δ {A}_{ac , out} = - \frac{{Δ I}_{a c} sin ( θ )}{{I}_{d c} ln ( 10 )} + \frac{{Δ I}_{ac , 0} sin ( {θ}_{0} )}{{I}_{dc , 0} ln ( 10 )} & ( 16 )\end{matrix}$$

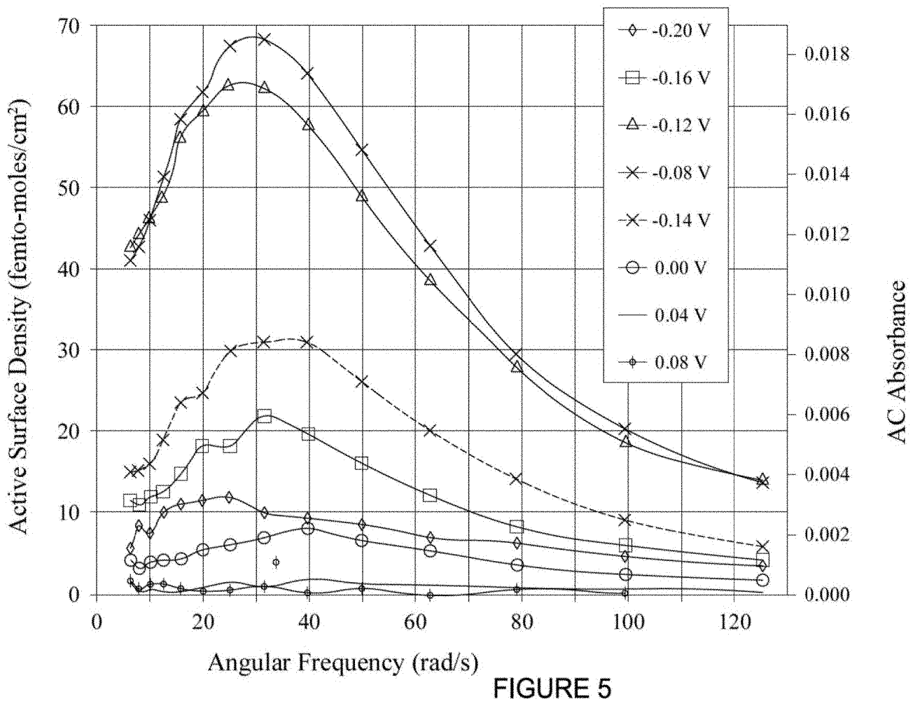

The quantities which are obtained then enable implementation of an optical impedimetric measurement based on the output of the device. FIG. 5 shows experimental results from impedance measurements for wave-guided light at 550 nm propagating along the device to interrogate a submonolayer of an electro-active redox Cytochrome c (Cyt-C) protein. Because Cyt-C has well-characterized spectroelectrochemical properties in solution, testing this protein served as a useful surrogate reporter for the spectroelectrochemical response after the EA-IOW was functionalized. Using this surrogate, the data taken at various potentials indicates a sufficiently strong analytical signal for detection at relatively low density levels. Additionally, the inventive EA-IOW's when used in the immunoassay context distinguish between different analyte species present in a solution, because they are designed to capture a particular analyte or antigen without binding to others. As an illustrative example only, in testing directed to Cyt-C detection over the capture antibody layer, hemoglobin (Hb) protein in the same sample was only negligibly adsorbed, a lack of interaction that indicates a selective design of the inventive EA-IOW biased toward analytes of interest.

The surface density, ΔΓ_{ac} of the electro-active species is determined from the measured data of the in-phase and out-of-phase components using equation 17:

$$Δ {Γ}_{a c} = \frac{\sqrt{{( Δ {A}_{ac , in} )}^{2} + {( Δ {A}_{ac , out} )}^{2}}}{SΔ ϵ} \qquad ( 17 )$$

In turn, from the surface density, ΔΓ_{ac}, of the electro-active species, one can determine the associated faradaic current density by using the relation expressed in equation (18)

Δi_{F}=nFΔΓ_{ac}ω (18)

The results from this relationship are expressed in terms of Faradaic information as shown in FIGS. 6A and 6B for Cyt-C. In FIG. 6B, each curve shows the characteristic formal potential and potential width of the redox processes for this electro-active species. Likewise, similar studies can be obtained with other electro-active species.

The data thus indicate that the measurements obtained with device 10 provide sufficient analytical signals that can be readily followed and measured, even when a small density of redox species (femto-moles/cm²=0.005% of a full protein monolayer=0.1 pg/mm²) are involved in the electron transfer process. Moreover, the optical data for other redox-labeled reporter antibodies will be amenable to reconstruct the electrochemical information of Faradaic processes associated with redox events involving these molecules.

### Spectroelectrochemical Detection of HA Protein

Now returning to FIG. 2, proximal the ITO layer is a redox probe 286 (an antibody or other protein) with an electro-active species 285 at its center, followed by redox processes, which can also be taking place while modulating the potential within the cell. As above stated, a sensitivity factor, S, of device 10 represents the signal enhancement provided when compared to either the single-bounce reflection or single-pass transmission. As discussed in an earlier section on fabrication, a sensitivity factor generally depends on the layers' thickness and refractive index, as defined by the ratio between the waveguide absorbance and direct transmission absorbance.

Accordingly, a device 10 is fabricated which offers high optical transparency for propagation of the light beam, and good electrical conductivity. Aspects of device 10 that tend to increase the sensitivity factor are its single-mode operation and the long propagation length (about 2.54 cm, in an embodiment) of the guided light beam along the device. The separation between the two gratings is represented by points 140 and 150, respectively, in FIG. 1A and FIG. 2, such that the distance between these points defines the propagation length along the device. In alternative embodiments, the propagation length is at least 1 mm. A light beam propagating for about 3.4 cm along the waveguide before outcoupling occurs via grating coupler 150 has been found acceptable. As explained further herein, the conductivity and sensitivity factor of a device 10 also is used for the detection of redox-active adsorbates even at low surface densities of redox species that bind to antigen, or weak molar absorptivities, or both.

As further shown in FIG. 2, a pair of grating couplers 140, 150 formed on a substrate 218 which can be glass or other transparent material, are integrated into the waveguide structure for coupling a light beam in and out of the wave guide and spectrally dispersing the light beam. In an embodiment, the gratings are formed on a surface of the substrate as part of the fabrication process by reactive ion beam etching. The photo-patterns are created through holographic exposure under suitable beam intensity, with the direction of the beam controlled by a Loyd's mirror configuration and under real-time monitoring of the development process. Further, a suitable pitch-size of the surface-relief gratings is about 323 nm, although other dimensions are also workable. A wavelength of about 530 nm is one of several suitable wavelengths for coupling a light beam in and out of device 10 via the waveguide gratings 140, 150 and associated optics (not shown). In an embodiment, the device 10 is fabricated with a propagation length of about 3 cm between gratings 140, 150, enabling optical monitoring of redox couples in connection with spectroelectrochemical transduction.

In view of the above teachings, the detection of hemagglutinin (HA) protein from the H5N1 influenza virus was accomplished. A monoclonal anti-H5 (H5N1) capture antibody specific to the H5N1 antigen underwent binding to a functionalized EA-IOW interface. This was followed by exposure to the virus antigen influenza A hemagglutinin protein (HA) at a concentration of 200 ng/mL. Exposure resulted in immobilization of HA through binding to the capture antibody, as evidenced by the adsorption of a polyclonal reporter Ab labeled with methylene blue (MB) ester dye, and which served as an redox-active probe.

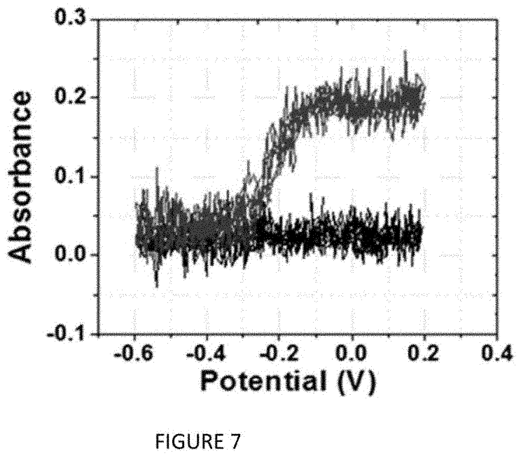

For the detection of an HA protein in a sample, FIG. 7 graphically depicts results from optical absorbance at 610 nm based on the presence of surface-bound methylene blue-reporter antibody using cyclic voltammetry (CV) scans in connection with an inventive EA-IOW according to present embodiments. As indicated by the legend, the tracings are indicative of both graphs the optical absorbance results at 610 nm based on cyclic voltammetry (CV) scans using a EA-IOW according to present embodiments for an EA-IOW functionalized with APTES and capture antibody. The separate tracings are shown for samples with HA virus antigen (lighter shading, upper tracings, conc. 200 ng/ML) and without virus antigen (darker shading, lower tracings), respectively.

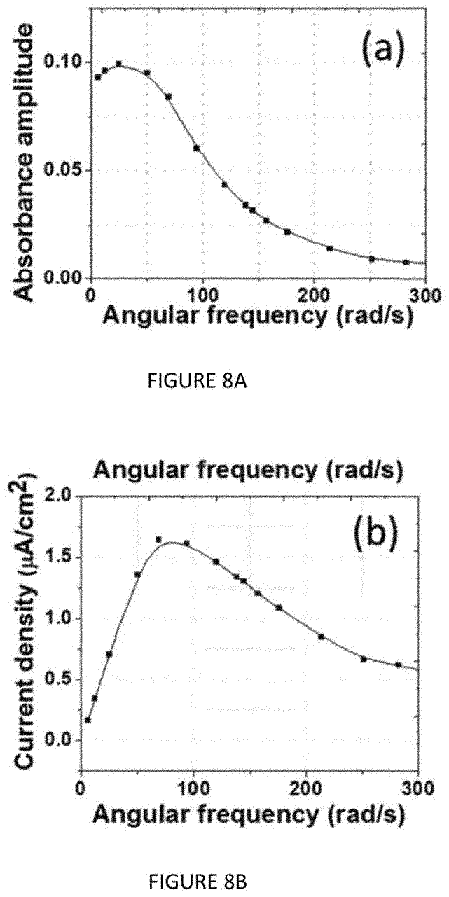

Now with respect to FIG. 8A, the absorbance amplitude was measured under AC potential modulation for a EA-IOW functionalized for a methylene blue-reporter antibody redox probe, at a potential modulation amplitude of 30 mV, DC bias potential of −220 mV, laser wavelength for the optical beam source of 633 nm, and finally a virus protein concentration of 200 ng/mL. The collected optical data then allow the corresponding faradaic current density to be determined, as referenced in FIG. 8B.

As the graph in FIG. 8B reflects, the faradaic current density features a peak value centered at about 50 rad/s, which is associated with the electron transfer rate of the redox event that occurs at the electrode surface of the EA-IOW. With this information concerning a desirable angular frequency (i.e., 50 rad/s), AC voltammetry was applied at different potentials while the EA-IOW (device 10) and components of system 110 generated and collected optical data. For these purposes, a potential modulation with an amplitude of 30 mV was used, and the DC bias potential was varied over a range of (−360 mV to +40 mV), encompassing the formal potential of the redox process of the probe being used. Accordingly, FIG. 8B uses the optical absorbance data from the EA-IOW to graph faradaic current density against angular frequency values as a function of response to the antigen-bearing sample. According to multiple embodiments and alternatives described herein, processor 122 of system 110 executes computer-readable instructions programmed to receive the data generated by the electrochemical interactions and optical signals occurring at device 10. In turn, the program instructions being executed are configured to account for various parameters such as refractive indices and thicknesses, to determine the spectroelectrochemical events involving the immobilized redox probe, and to associate those findings with the presence of analyte or antigen specific to the reporter antibody.

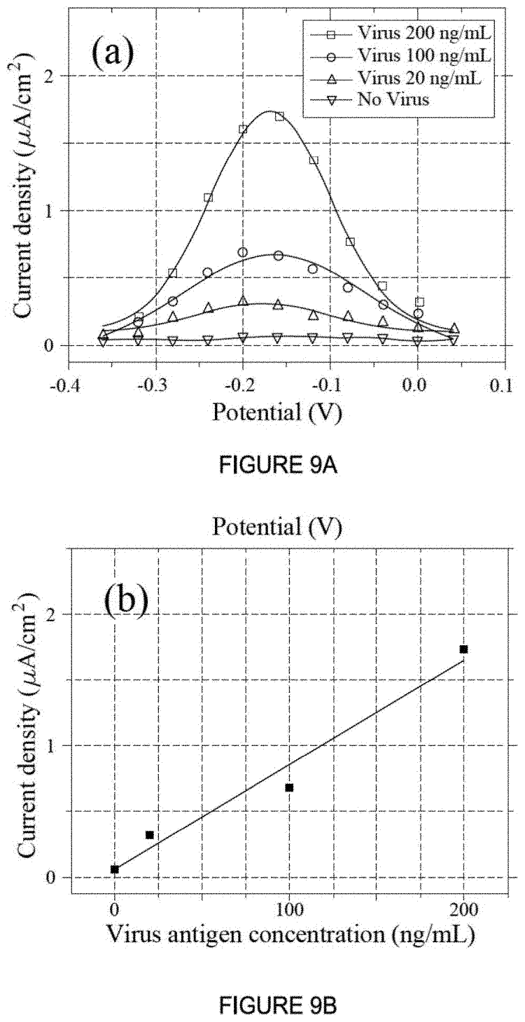

Quantification of the detected analyte is now discussed in view of FIG. 9A and FIG. 9B. In FIG. 9A, a plot of faradaic current density against the DC bias potential displays a maximum intensity at approximately −170 mV. As the DC bias potential is detuned from the formal potential (away from −170 mV) of the probe, the analytical signal decreases towards zero. The peak intensity of the faradaic current density reported by the redox probe is thus proportional to the surface concentration of the target antigen and provides a direct route to the quantification of the virus analyte.

As shown in FIG. 9B, when different bulk concentrations of the virus antigen solution were run, the AC voltammetric data enabled a determination of a limit of detection. This was done by taking the maximum of the faradaic current density for each concentration of virus antigen, based on laser wavelength, 610 nm; angular frequency, 50 rad/s; and amplitude modulation, 30 mV. By plotting the corresponding peak current density against the different bulk concentrations of virus antigen solution, a sub-100 pico-molar (pM) limit of detection was determined (i.e., a concentration of about 4 ng/mL), which surpasses several known detection techniques in use. The graphical data thus confirms the redox probe binds effectively with the HA virus antigen immobilized on the device surface, and is useful in detection and quantification. Beyond this, the low limits of detection are important in early detection of influenza.

Further, by functionalizing the EA-IOW surface according to the principles outlined herein, the inventive EA-IOW is customizable to detect and quantify many other infectious diseases and conditions other than influenza. Such tailoring will involve selecting a capture antibody suitable for immobilizing through binding a particular virus antigen, pathogen, or other analyte of interest; and exposing the array to a reporter antibody/redox probe having unique spectral and electrochemical properties that are detected when binding between the reporter antibody and the analyte occurs.

### Metallic Bilayer Structure for Enhanced Sensitivity in Redox Detection Applications of Electrochemically-Controlled Plasmonic Waves

Surface plasmon resonance (abbreviated as “SPR” herein) is one technique which has been used for its high sensitivity for characterizing and studying organic and inorganic thin films. Currently, the development of SPR is geared towards the design of compact, low-cost, and sensitive biosensors. Furthermore, the use of SPR is increasingly popular in fundamental biological studies, health science research, drug discovery, clinical diagnosis, and environmental and agricultural monitoring.

SPR allows for the qualitative and quantitative measurements of biomolecular interactions in real-time without requiring a labelling procedure. Although SPR is sensitive to the refractive index changes at the sensor surface, it still lacks sensitivity to probe redox species with weakly absorbing chromophores and/or at low surface concentrations. Another limiting factor for SPR is the need for a sufficiently high optical signal (ΔI) based upon refractive index changes associated with a redox adsorbate under interrogation. As discussed herein, current embodiments provide advantages by providing a sufficient EC-SPR response (based on the aforementioned optical signal) in cases of low analyte surface densities that are below detection levels when conventional detection systems are used.

An alternative to SPR that has been recently proposed is electrochemical surface plasmon resonance (abbreviated herein as “EC-SPR”) technology. Using this technique in imaging mode, the mass sensitivity has improved for detecting small molecules (e.g. <100 Da) and for determining the kinetic parameters of biomolecular binding events at the device active interface. Moreover, EC-SPR has shown the ability to study local electrochemical phenomena, such as the electrochemical activities of nanomaterials which is critical for creating novel catalysts, for developing ultrasensitive sensors, and for understanding fundamental nano-electrochemistry. However, despite the high sensitivity and the desirable limits of detection using the conventional EC-SPR for a broad spectrum of sensing applications, the background signal that comes from thermal noise, mechanical vibration, and non-specific adsorption events has a negative impact on detection sensitivity.

Accordingly, the descriptions and embodiments provided for herein are directed to a EC-SPR strategy for immunosensing detection. This principle is based on the detection of an optical signal generated by an electroactive redox mediator that is electrically-modulated by AC voltammetric modulation and strictly confined to the analytical interface. This methodology is both selective and specific as the monitored analytical signal is both optically and electrochemically locked to the spectroelectrochemical fingerprints of either naturally existing or specifically designed biomarkers for detection. It is optically locked by using a laser wavelength that is tuned to maximize the change occurring in the particular redox process. It is electrochemically locked by modulating the applied potential at an accessible formal potential of the redox probe and by the chosen modulation frequency of the aimed redox events. It is also locked to surface events next to the electrode interface as the surface plasmon resonance (“SPR”) wave generally is confined to surface penetration at the analytical interface of detection systems described herein. These factors have contributed desirably to reducing the effects of background signals from possible interference from constituents in the bulk solution, circumventing non-specific detection events, and tracking the targeted redox event during detection.

Further, the present descriptions and embodiments overcome a number of challenges previously observed in this field, in relation to fabrication. Such challenges have resulted in narrower spectral ranges of plasmonic surface waves, which could be utilized, allowing only a small number of available redox couples and narrower spectrochemical applications. In the traditional EC-SPR approach, for example, a gold layer is typically used for plasmonic surface waves. However, due to the intrinsic optical properties of gold, only wavelengths longer than 630 nm can be used in the conventional EC-SPR approach. This is a limiting factor because it excludes many redox couples from being used in the immunosensing detection at wavelengths shorter than 630 nm. Likewise, a single layer of silver is less desirable because it is chemically degraded under electrochemical modulation in an aqueous environment.

Accordingly, the descriptions and embodiments provided for herein meet a significant need for EC-SPR strategies that support a broader spectral range of plasmonic surface waves (i.e. wavelengths shorter than 630 nm) which would permit a broader range of available redox couples to be deployed for biological and chemical detection. Other challenges overcome by the descriptions and embodiments herein include the effects of background signals in the biosensors technology, which make it more difficult to detect and characterize molecular assemblies with low surface densities. In accordance with the present descriptions and embodiments herein, a user can investigate a variety of redox-transduced events with small differences in their molar absorptivities, and facilitate easier and more convenient fabrication. Along with other features and advantages outlined herein, the methods and bimetallic structures within the scope of present embodiments meet these and other needs. In doing so, the methods and structures, according to multiple embodiments and alternatives, optimize the spectroelectrochemical transduction imprinted into the optical signal at low surface densities, optically monitor a much broader range of redox couples (even those with weak optical changes under redox events) by supporting wavelengths shorter than 630 nm, broaden the range of available spectrochemical applications, and are easier to fabricate by requiring fewer components (e.g. no grating coupler).

In some embodiments, related to EC-SPR, an electrochemical detection system comprising a light source that generates light waves and a coated substrate (which may be located in a flowcell containing an operating environment for receiving an electroactive probe). For example, the operating environment may be an aqueous environment. Also, in some embodiments the coated substrate comprises a first layer which is a silver (Ag) layer and a second layer which is a gold (Au) layer to form a bilayer, and wherein the gold (Au) layer is arranged to isolate the silver (Ag) layer from the operating environment, so that the aqueous liquid inside the flowcell or other housing containing the bilayer does not contact the silver layer. In some embodiments, the light source is a continuous wave laser, such as a surface plasmon wave device as described herein.

As discussed below, in an exemplary process SPR reflectance curves and a cyclic voltammetry (“CV”) technique were utilized to create a desirable SPR configuration comprising a silver and gold bilayer structure for enhanced sensitivity in redox detection applications. A well-characterized redox-active probe, the cytochrome c protein, was utilized as a non-limiting example. Based on the experimental data discussed below from the use of the cytochrome c protein, it is also expected that the redox couples nile red, nile blue, viologen, and anthraquinone (as non-limiting examples) are expected to be useful at wavelengths shorter than 630 nm with the methods and structures within the scope of present embodiments. Moreover, without the improvements gained by the methods and structures within the scope of present embodiments, it is expected that these redox couples would not have been available because the conventional approaches could only support longer wavelengths and not those below 630 nm.

### Description of an Exemplary Process and Results

Reagents and Device Configuration—Cytochrome c protein (20 μg/ml, 1 ml, molecular weight=144 kDa) from horse heart with 99.7% purity; sodium phosphate monobasic and sodium phosphate dibasic were used to prepare a 50 mM solution of phosphate buffered saline (PBS); 3-Mercaptopropionic acid (MPA); ethanol (95%); and acetone (>99.5%).

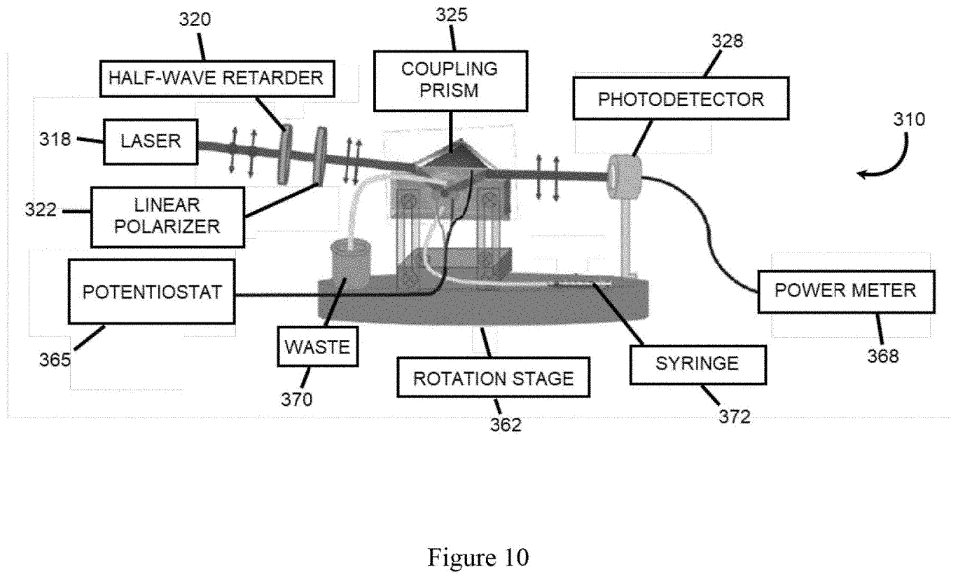

FIG. 10 generally illustrates a EC-SPR system 310 for an immunoassay as described herein. In the exemplary process, the system 310 consisted of two components: an optical component 312 and a micro-electrochemical flowcell 350. The optical component 312 comprised a SPR 315 that was based on the widely used Krectchmann-Raether configuration, which utilizes a prism-coupler or a diffraction grating coupler. The flowcell 350 was a three-electrode configuration, wherein either a single Au film or a bilayered film (Au/Ag) served as a working electrode (WE) 352, while two gold-plated pins (surface area=2.85 mm²) (MOUSER ELECTRONICS®, Mansfield, Tex., USA) were used as a reference electrode (RE) 355 and a counter electrode (CE) 358, respectively. The device, generally illustrated in FIG. 10, was employed for angular reflectance experiments to measure the optical constants of the Au, Ag, and MPA films. A continuous wave laser 318 (OBIS from COHERENT®, Santa Clara, Calif.) was deployed in combination with a half-wave plate retarder 320 and a linear polarizer 322 to establish linearly polarized light with transverse-magnetic (TM) polarization. The laser beam from laser 318 was directed towards the entrance facet of a coupling prism 325 to excite the surface plasmon wave; and five wavelengths (552 nm, 637 nm, 660 nm, 685 nm, and 785 nm) were used to excite the surface plasmon wave.

For the characterization measurements, the equilateral glass prism 325 (BK-7) was optically coupled to the metal-coated substrate using an index matching gel (n_{D}=1.52, CARGILLE®, Cedar Grove, N.J.). The device interface 360 was mounted in the micro-electrochemical flowcell 350 and then was mounted on rotation stage 362 (QUADRA-CHEK® 100) to precisely control the incident angle. The rotation stage 362 allows control of the incident angle, thus of the quantity nsin(θ), which determines the reflected power. The reflected optical intensity at different angles of incidence was directed to photodetector 328 and monitored by a power meter 368 (NEWPORT™, model 1930C).

For optimization of the analytical signal of the EC-SPR system 310, in the exemplary process a potentiostat 365 (CHI 660D, CH Instruments, Inc.) was connected to the WE 352, RE 355, and CE 358 to apply and control the potential at the WE. Two measurements that were conducted and are discussed in more detail below, are summarized as follows: in the first measurement, the SPR reflectance curves for a 500 nM cytochrome c protein solution were determined at two applied potentials (−0.3 and 0.3 V), corresponding to the oxidized and the reduced states of cytochrome c proteins respectively. The second set of measurements were taken under cyclic voltammetry (CV) modulation. For these measurements, a potential modulation from (−0.3 V to 0.3 V) at a scan rate of 0.02 V/s was applied, while the electrically modulated reflected optical signal was monitored at a fixed angle by the power meter 368 and transferred to an oscilloscope (not shown) (AGILENT®, DSO8104A Infiniium). FIG. 10 also illustrates waste receptacle 370 and syringe 372.

Functionalization of the Bilayered Surface with the MPA—In the exemplary process, the device interface 360 of the bilayered (Au/Ag) coated glass slide was sequentially cleaned in an ultrasonic bath with deionized (DI) water, acetone, and ethanol for 10 minutes in each solution, and then was dried under N₂ gas. After these steps, the device interface 360 was immersed into a 10 mM solution of MPA in ethanol for 3 hours to establish a self-assembled monolayer (SAM) on the device interface 360. After rinsing the surface with ethanol and DI water, and drying it gently with N₂ gas, the MPA-coated surface was mounted on the flowcell 350 and stabilized with the 50 mM PBS buffer solution under CV potential modulation from (−0.3 V to 0.3 V) at scan rate of 0.02 V/s.

### Results and Discussion

Characterization of the Thin Films—The system 310, generally shown in FIG. 10, was employed for angular reflectance experiments to measure the optical constants of the Au, Ag and MPA films. Simulation results were used to match the measured SPR reflectance curves and obtain the optical constants of each layer by minimizing the difference between the experimental and the simulated data. Five excitation wavelengths (552, 637, 660, 685, and 785 nm) were used.

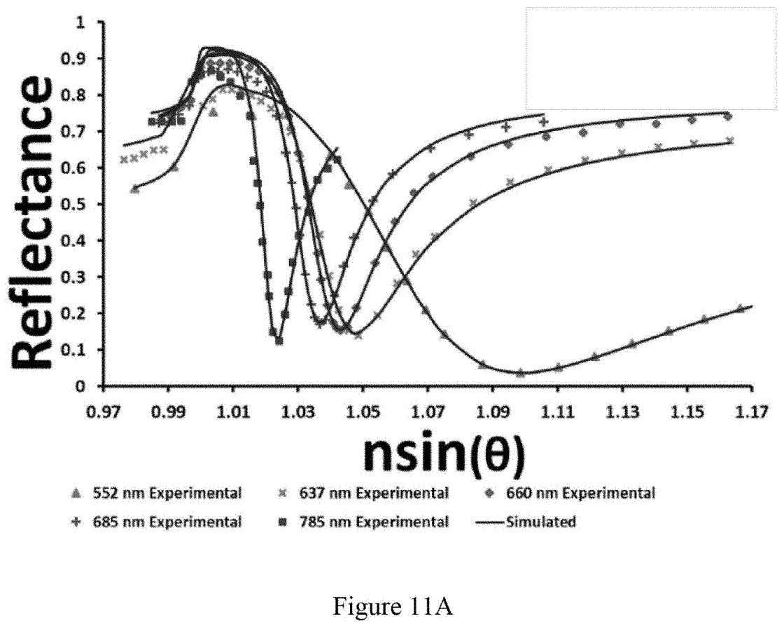

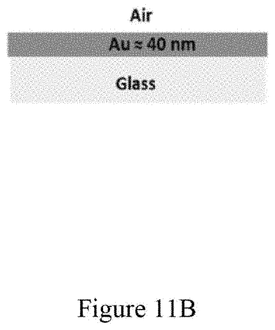

Measurement of the Gold Thin Film—Cleaned microscope slides were coated with a 45 nm Au film using a dc magnetron sputtering technique (Nano-Master) and mounted on the flowcell 350. Then the incident angle was scanned and the reflected optical intensity at different angles of incidence was monitored. For the simulated data, a three-layer (BK7/Au/air) model was used as illustrated in FIG. 11B. However, FIG. 11B illustrates a 40 nm Au film which was used in this experiment for preliminary characterization of the gold film, and a 45 nm Au film was utilized for further optimization of performance. The measured and simulated SPR reflectivity curves for 45 nm Au thickness in air and the five excitation wavelengths are shown in FIG. 11A. In FIG. 11A, the dots illustrate the experimental results obtained by the conventional SPR setup at each excitation wavelength and the solid lines are the simulated SPR reflectivity curves. As previously noted, the wavelengths of light used were 552 nm (represented by the triangles in FIG. 11A), 637 nm (represented by the “x” symbol), 660 nm (diamonds), 685 nm (plus signs), and 785 nm (squares).

Table V lists the fitting parameters for the Au layer at each excitation wavelength. The optical constant of the Au, Ag, and MPA films were extracted by minimizing the difference between the measured SPR reflectance curves and the simulated curves. Herein, the letters “n” and “k” refer to the real and imaginary parts, respectively, of the refractive index of the material. For MPA, the k-value is assumed to be zero which is a feature of a transparent material.

TABLE V
Fitting Parameters

| Wave- | | | | | |
|---|---|---|---|---|---|
| length | Ag | | Au | | MPA |
| (nm) | n | k | n | k | n |
| 552 | 0.0901 | 3.332 | 0.38559 | 2.61439 | 1.5313 |
| 637 | 0.1307 | 4.3535 | 0.208201 | 3.734 | 1.5569 |
| 660 | 0.152 | 4.6014 | 0.159619 | 3.82814 | 1.5524 |
| 685 | 0.1611 | 4.7767 | 0.155341 | 4.15894 | 1.542 |
| 785 | 0.1762 | 5.5224 | 0.193676 | 5.28401 | 1.4839 |

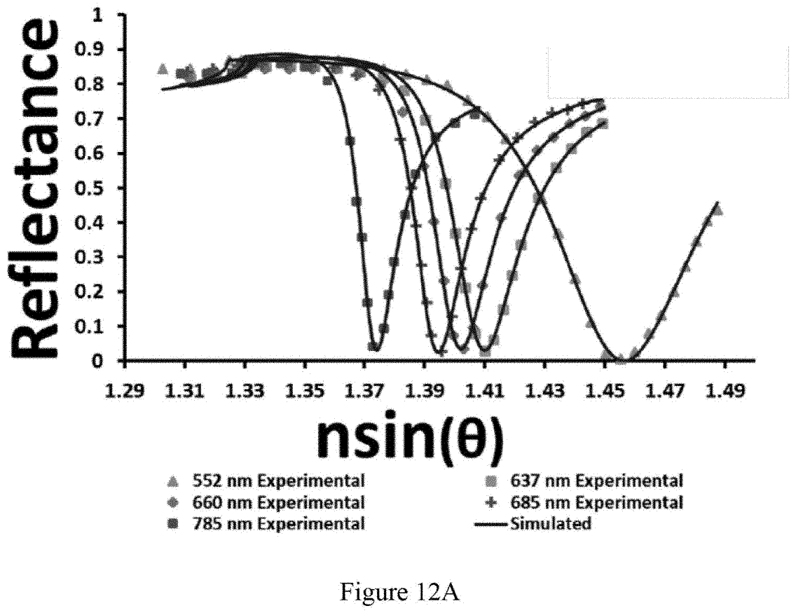

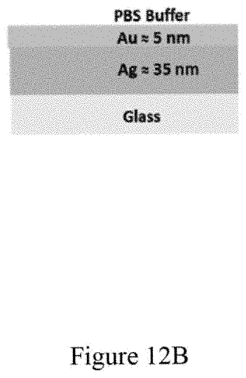

Measurement of the Optical Constants of Silver Thing Film—In the exemplary process, a bilayered (Au/Ag) film with about 5 nm thickness of Au and about 35 nm thickness of Ag (abbreviated as 5/35 nm herein), was used to determine the optical constant of an Ag film. An operating environment for this exemplary process was an aqueous environment. As will be appreciated from the current example, in some embodiments thicknesses of the silver and gold of the Ag—Au bilayer are determined to provide strong transfer of energy from an incoming light wave into a propagating surface wave travelling on the device surface. For example, a thickness of the silver (Ag) layer can range from 10-60 nm and a thickness of the gold (Au) layer from 2-50 nm. For the simulated data, a four-layer (BK7/Ag/Au/PBS buffer) model was used, as illustrated in FIG. 12B. The measured and simulated SPR reflectivity curves for bilayered (Au/Ag) film using the five excitation wavelengths are shown in FIG. 12A. FIG. 12A illustrates the optical constants examination of the Ag layer in the PBS buffer, where the dots are the experimental results obtained by a SPR setup at each excitation wavelength and the blue solid lines are simulation SPR reflectivity curves. In FIG. 12A, the excitation wavelengths are 552 nm (represented by the triangles in FIG. 12A), 637 nm (lighter squares), 660 nm (diamonds), 685 nm (plus signs), and 785 nm (darker squares). The fitting parameters for the Ag layer at each excitation wavelength are listed in Table V (above).

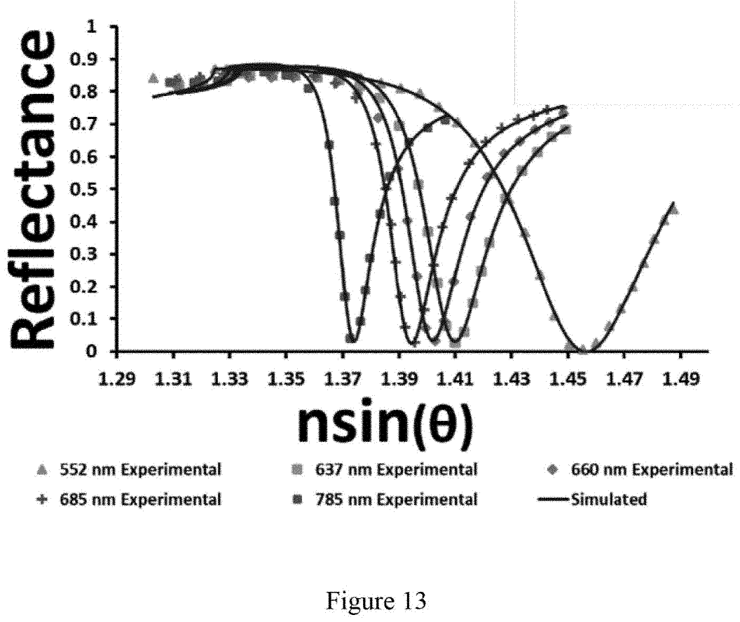

Measurement of the Optical Constants of the MPA Film—The system 310, generally illustrated in FIG. 10, was used to measure the SPR curves for MPA-bilayered (Au/Ag) films using the previously noted five excitation wavelengths. For the simulated data, a five-layer (BK7/Ag/Au/MPA/PBS buffer) model was used, in which the MPA layer has a suitable thickness to immobilize each protein-containing redox probe that is being used to detect the reduction/oxidation signal changes. In some embodiments, MPA is in the form of a molecular monolayer. As noted above, the optical constants of the Au and Ag films were previously determined. The measured and simulated SPR reflectivity curves of the MPA layer in a PBS buffer, using the five excitation wavelengths, are shown in FIG. 13. The data points are the experimental results obtained with a SPR setup at each excitation wavelength and the blue solid lines are simulated SPR reflectivity curves. In FIG. 13, the wavelengths of light are 552 nm (represented by the triangle symbol), 637 nm (lighter square), 660 nm (diamond), 685 nm (plus sign), and 785 nm (darker square). The optical constants of the MPA layer at the five excitation wavelengths are listed in Table V (above).

Optimization of the Analytical Signal of a EC-SPR System—An EC-SPR system relies on a strong difference in the optical response between the reduced and oxidized states of a redox-active probe (commonly abbreviated as ΔI). In the exemplary process, the cytochrome c protein redox probe was used to search for a desirable SPR device configuration to optimize the EC-SPR response with the highest optical signal ΔI.

Three different configurations in an aqueous environment were used: Au, Ag, and bilayer (Au/Ag) films. The reflectivity under angular interrogation with five excitation wavelengths (552 nm, 637 nm, 660 nm, 685 nm, and 785 nm) and different thicknesses were simulated based on transfer matrix method—a known mathematical formalism based on certain laws of Physics (i.e., Maxwell's equations) that allows the calculation of the fraction of power of the reflected, transmitted and absorbed light waves, for light incident on a device under a specific incident angle and defined light polarization. In general, the Ag film performs better in that it yields a sharper and narrower SPR dip profile than both the Au film and the bilayer (Au/Ag) film for all excitation wavelengths. Despite the Ag film producing a narrower SPR dip profile, the Ag film was expected to have a lower chemical stability, so a bilayered (Au/Ag) film with approximately 5/35 nm (Au/Ag) thickness was selected in an exemplary process with the Ag film improving the SPR sensitivity and the Au film providing chemical stability for the SPR platform.

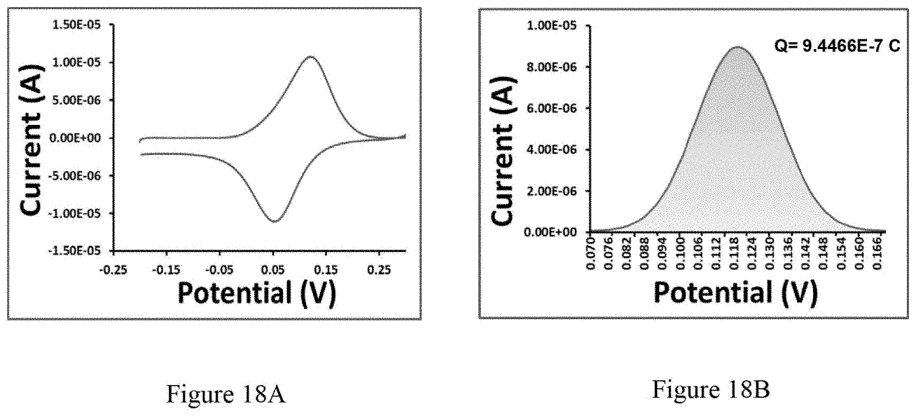

For the full optimization tests of the EC-SPR system, the MPA-bilayer (Au/Ag) surface was mounted into an electrochemical cell, otherwise the configuration as illustrated in FIG. 10 was utilized. A monolayer of cytochrome c proteins was deposited onto the MPA-bilayered (Au/Ag) surface by injecting a 50-μL solution of cytochrome c proteins at a concentration of 500 nM in the flowcell 350 and incubating for 30 minutes. Then the flowcell 350 was thoroughly rinsed with a PBS solution to remove unbound species. A mono-layer of cytochrome c proteins was immobilized on the surface by electrostatic binding to a MPA self-assembled monolayer (SAM). As shown in FIGS. 18A and 18B, a cyclic voltammetry (CV) technique was used to confirm the mono-layer coverage. In particular, FIG. 18A illustrates the cyclic voltammograms with cytochrome c proteins immobilized onto the MPA SAM preformed onto a bilayered Ag/Au surface with scan rate of 0.02 V/s. FIG. 18B illustrates the redox peak which was measured using Equation (19):

$$Γ = \frac{Q}{nFA} = 11.66 pm / {cm}^{2} \qquad ( 19 )$$

The measured redox peak area of 11.66 pmol/cm² is very close to the calculated theoretical maximum coverage for cytochrome c proteins on gold surface (Γ=15 pmol/cm²).

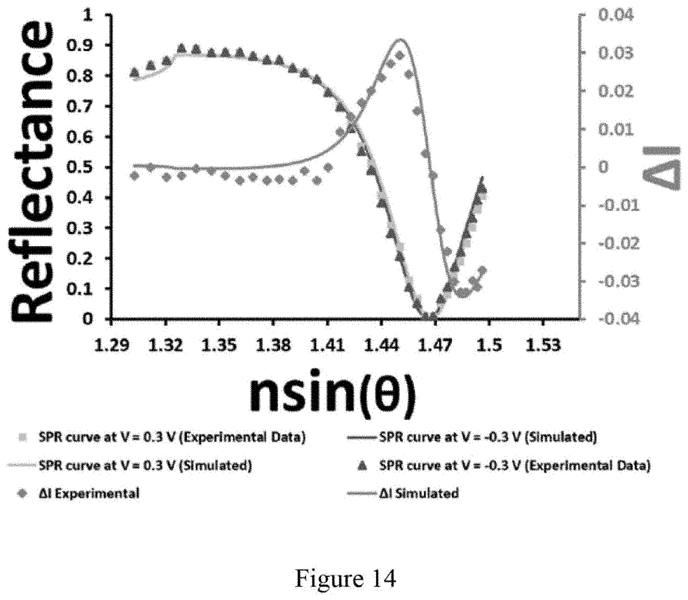

Typical SPR Reflectance Curve Measurements—In the exemplary process, the following two electric potentials were applied to control the oxidation states (and therefore the optical response of the cytochrome c proteins): −0.3 V (which corresponds to a reduced state of cytochrome c proteins) and 0.3 V (an oxidized state of cytochrome c proteins). The SPR reflectance curves for each oxidation state were then measured. Next, simulation results based on a commercially available technical computing system program (Wolfram Mathematica®, Wolfram Group, LLC, Champaign, Ill.) were used to match the measured SPR reflectance curves to determine the optical constants of the cytochrome c proteins in both oxidized and reduced states. For the simulation data, a six-layer (BK7/Ag/Au/MPA/cytochrome c proteins/PBS buffer) model was used. FIG. 14 illustrates the measured and simulated SPR reflectance curves against the effective refractive index inside the prism 325 for a 500 nM cytochrome c protein solution using a 552 nm excitation wavelength. In FIG. 14, the darker triangular dots and darker solid line represent the experimental results at −0.3V, while the lighter squares and lighter solid line are the results at 0.3V. The curve with diamond symbols shows the experimental measurement of ΔI, and the solid curve is the theoretical calculation of ΔI.

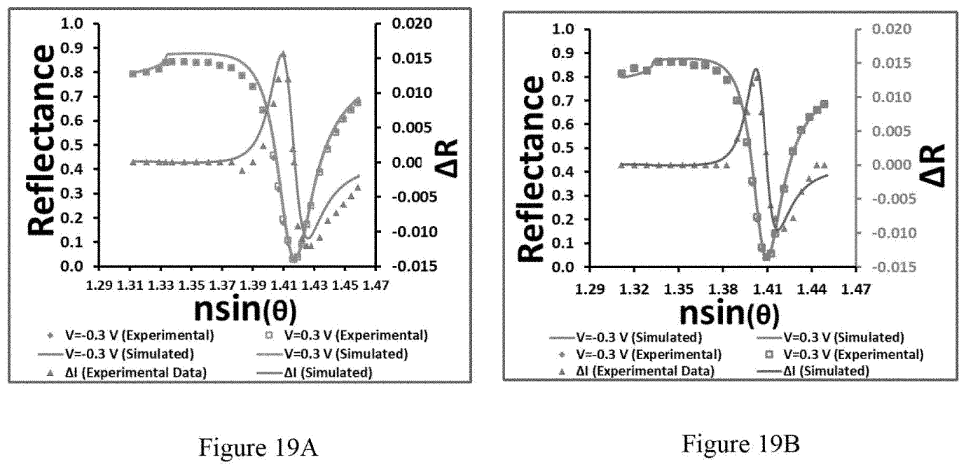

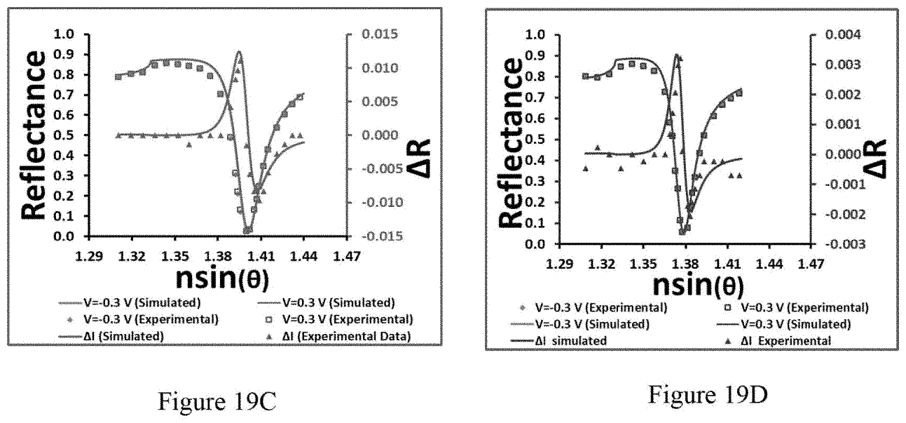

The same procedure was conducted using four excitation wavelengths (637 nm, 660 nm 685 nm, and 785 nm) and the results of the optical constants examination of the cytochrome c layer in PBS buffer are shown in FIGS. 19A-19D. FIG. 19A illustrates the examination using a 637 nm excitation wavelength, FIG. 19B used a 660 nm excitation wavelength, FIG. 19C a 685 nm excitation wavelength, and FIG. 19D a 785 nm excitation wavelength. In addition, the graphs in FIG. 19A-19D illustrate the experimental results obtained at −0.3V and 0.3V, as well as the simulation SPR reflectivity curves using the six-layer structure (BK7/Ag/Au/MPA/cytochrome c proteins/PBS buffer).

The optical constants of the cytochrome c proteins at the two applied potentials were then determined by minimizing the difference between the measured SPR reflectance curves and the simulated curves, and the constants are listed in Table VI below.

TABLE VI
Optical Constants of Cytochrome C

| Wave- | Oxidized | | Reduced | |
|---|---|---|---|---|
| length | cytochrome c | | cytochrome c | |
| (nm) | n | k | n | k |
| 552 | 1.5255 | 0.0026 | 1.5655 | 0.009 |
| 637 | 1.5789 | 0.0005 | 1.5909 | 0.00043 |
| 660 | 1.5793 | 0.00045 | 1.5937 | 0.00035 |
| 685 | 1.5359 | 0.00045 | 1.547 | 0.00035 |
| 785 | 1.614 | 0.00023 | 1.6904 | 0.00038 |

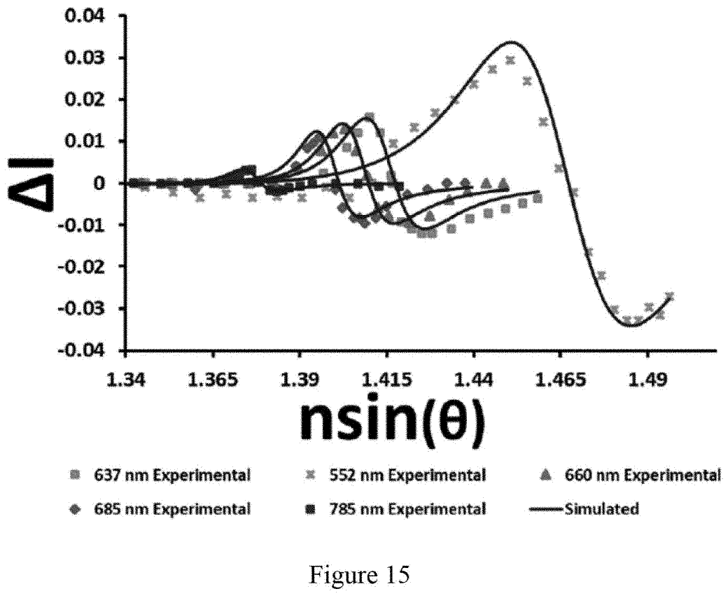

Next, the optical signal difference (ΔI), was determined by subtracting the reflectance of the cytochrome c proteins solution at 0.3 V from the reflectance of the cytochrome c proteins solution at −0.3 V. As shown in FIG. 14, the highest magnitude of the ΔI was observed in the linear region on the left side of the resonant angle of the conventional SPR curve, and the experimental data (the curve with diamond symbols) was in excellent agreement with the theoretical calculations (solid line curve). Using these experimental and simulated results, the experimental and simulated optical signal differences, ΔI, were plotted against the effective refractive index inside the prism [nsin(θ)] at 552 nm, 637 nm, 660 nm, 685 nm and 785 nm excitation wavelengths, as shown in FIG. 15. In FIG. 15, the dotted curves are the experimental data and the solid curves are the theoretical calculations.

The experimental and simulated curves illustrated in FIG. 15 show that the highest magnitude ΔI was obtained by using 552 nm as the excitation wavelength, where the ΔI relies on the change in both the real and imaginary parts of the refractive index of the cytochrome c proteins when undergoing a redox process. Since the actual optical transition of the iron center in cytochrome c proteins is responsible for both the redox potential and the optical change has a high absorbance light near 550 nm, the conclusion drawn from this data, without intending to be limiting on the scope of embodiments herein, is that tuning the excitation wavelength to the absorption band of the redox probe will maximize the amplitude of the optical modulation when an electrochemical modulation is applied.

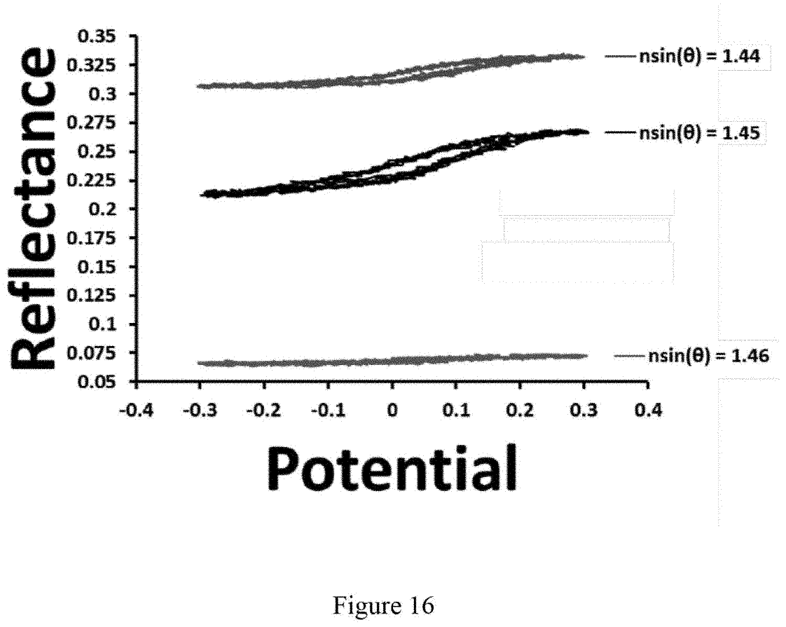

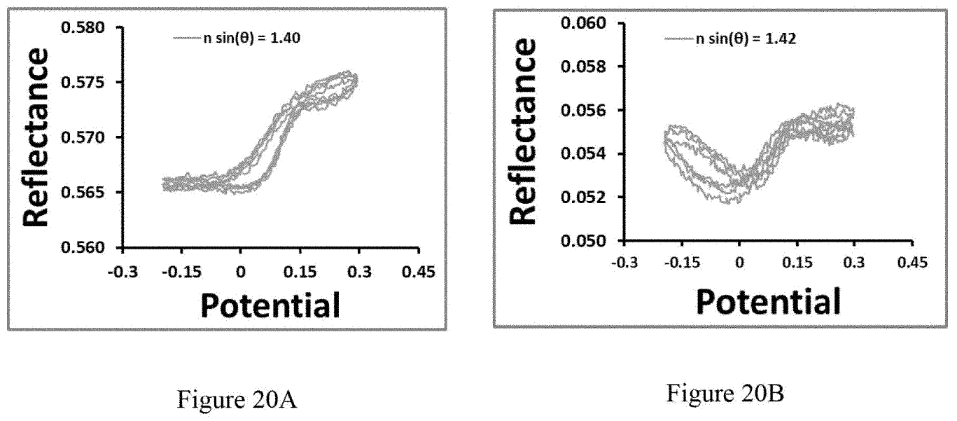

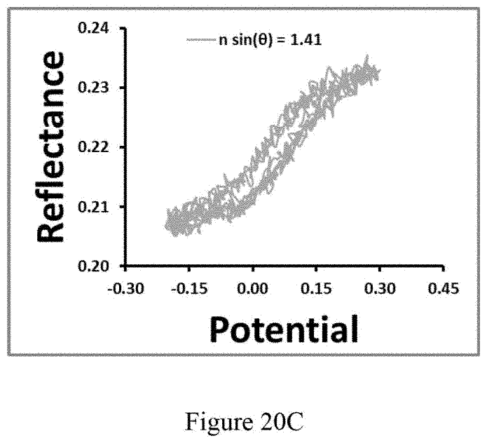

Cyclic Voltammetry (CV) Measurements—To demonstrate the results in the previous section, a series of potential scans were conducted to the cytochrome c proteins immobilized onto a MPA preformed onto a bilayered (Au/Ag) surface from (−0.3 to 0.3 V) at a scan rate of 0.02 V/s. In the exemplary process, three values of nsin(θ) were examined for each excitation wavelength: 1.44 (red trace), 1.45 (black trace), and 1.46 (blue trace). The results from the CV scan are shown in FIG. 16 and the results using the wavelengths 637 nm, 660 nm, 685 nm and 785 nm are shown in FIGS. 20A-23C (discussed in more detail below).

FIG. 16 shows a reversible change in the difference in the reflectance as the modulation potential crosses the formal potential of the cytochrome c proteins (approximately at 0.05 V). From this data, the ΔI were estimated to be 0.54 for black trace (nsin(θ)=1.45), 0.25 for red trace (nsin(θ)=1.44) and 0.006 for the blue trace (nsin(θ)=1.46).

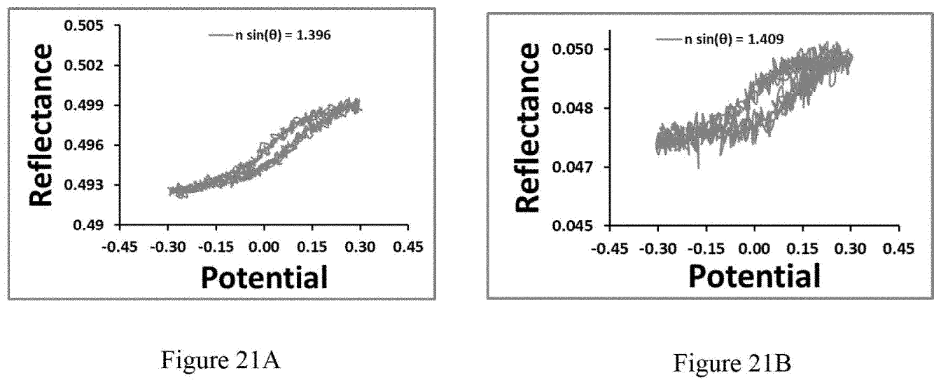

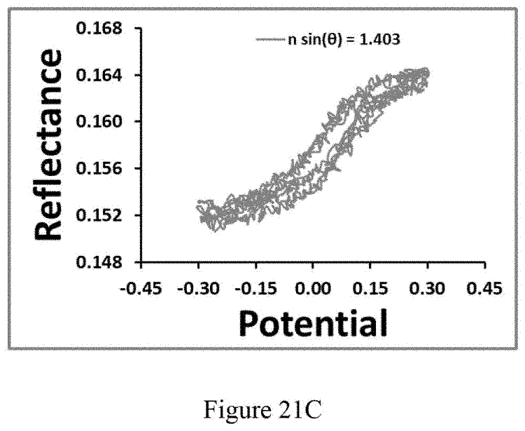

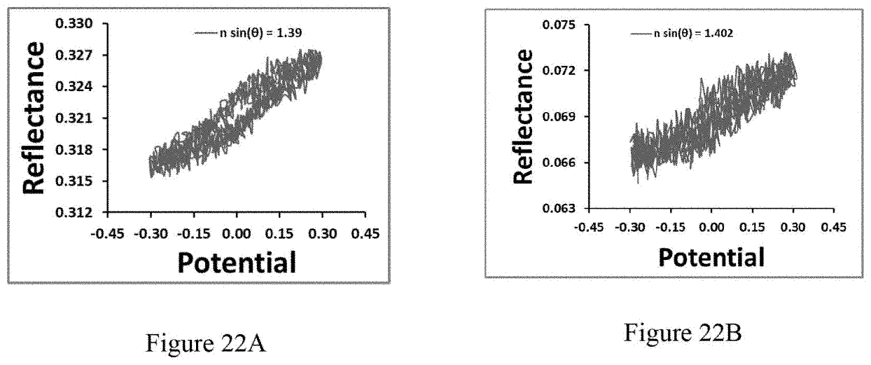

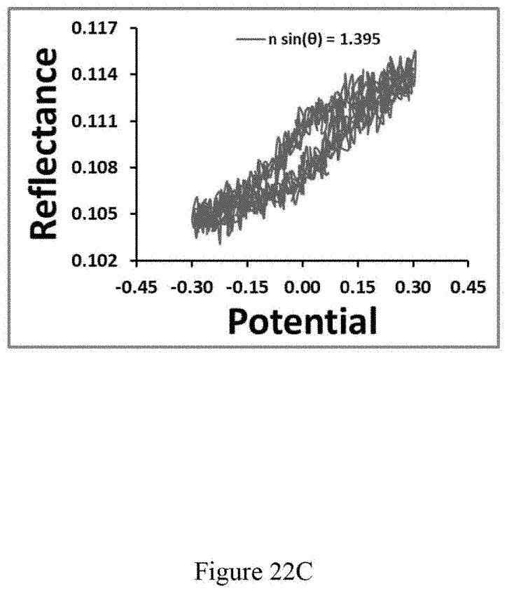

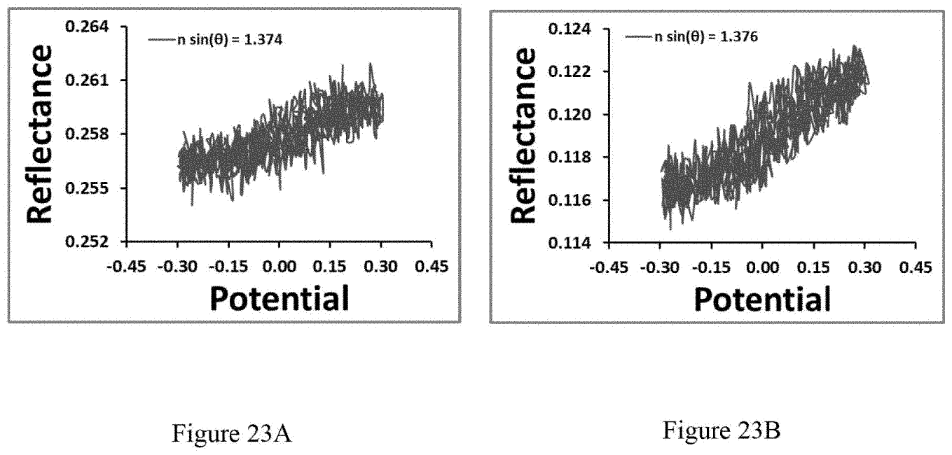

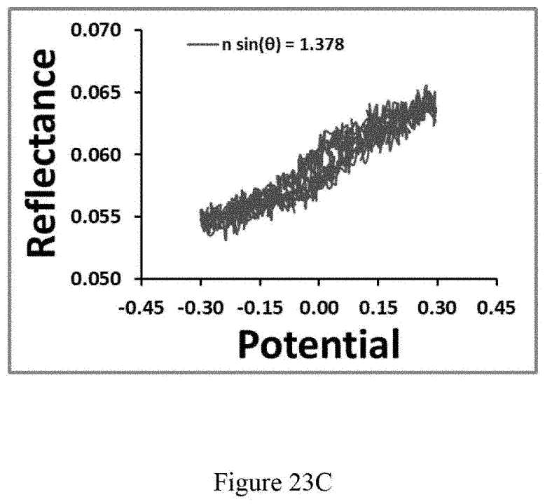

As shown in FIG. 20A-23C, the same trend was also observed with the other wavelengths. FIGS. 20A-20C illustrate the reflectance under a CV scan for the cytochrome c protein immobilized onto a MPA preformed onto a bilayered (Au/Ag) surface with a scan rate of 0.02 V/s and at three values of n sin(θ) for a 637 nm excitation wavelength. FIGS. 21A-21C illustrate the reflectance under a CV scan for the cytochrome c protein immobilized onto a MPA preformed onto a bilayered (Au/Ag) surface with a scan rate of 0.02 V/s and at three values of n sin(θ) for a 660 nm excitation wavelength. FIGS. 22A-22C illustrate the reflectance under a CV scan for the cytochrome c protein immobilized onto a MPA preformed onto a bilayered (Au/Ag) surface with a scan rate of 0.02 V/s and at three values of n sin(θ) for a 685 nm excitation wavelength. Lastly, FIGS. 23A-23C illustrate the reflectance under a CV scan for the cytochrome c protein immobilized onto a MPA preformed onto a bilayered (Au/Ag) surface with a scan rate of 0.02 V/s and at three values of n sin(θ) for a 785 nm excitation wavelength.

Accordingly, the results illustrated in FIGS. 16 & 20A-23C show that the magnitude of the useful reflectance ΔI at certain excitation wavelengths is highly dependent on the nsin(θ), and the highest magnitude of the ΔI is observed in the linear region on the left side of the resonant angle of the conventional SPR curve.

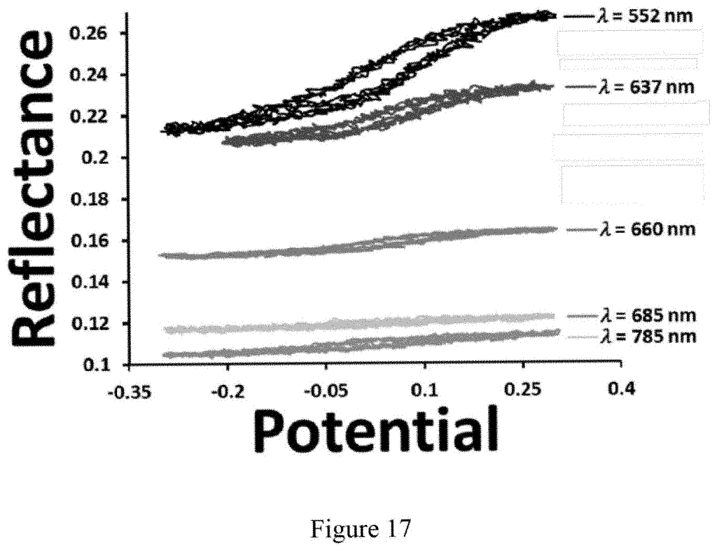

Next, in order to determine the dependence of the ΔI on the excitation wavelength, the CV measurements at a nsin(θ) providing a desirable magnitude of the ΔI for the five excitation wavelengths were plotted, as shown in FIG. 17. FIG. 17 shows the reflectance under a CV scan for the cytochrome c proteins immobilized onto an MPA SAM preformed onto a bilayered (Au/Ag) surface with a scan rate of 0.02 V/s. Further, the measurement shown in FIG. 17 used a desirable nsin(θ) at the following wavelengths: 552 nm (black trace), 637 nm (red trace), 660 nm (blue trace), 685 nm (yellow trace), and 785 nm (orange trace). The nsin(θ) was different for each wavelength and optimized the signal for that particular wavelength.

The data in FIG. 17 indicate that the highest magnitude of the useful ΔI was observed for the excitation wavelength 552 nm (black trace), whereas the smallest ΔI was observed in the excitation wavelengths 685 nm and 785 nm. These results are in excellent agreement with the results determined in the previous section, and further show that the ΔI is largely affected by the nsin(θ) and the excitation wavelength, therefore defining the optimal nsin(θ) and the wavelength to implement EC-SPR measurements in an effective way to enhance and increase the sensitivity of EC-SPR system.

In the exemplary process, a cytochrome c protein immobilized on an MPA-bilayered (Au/Au) surface was used to generate the desirable parameters of the EC-SPR system 310 which maximizes the difference of the optical response between the reduced and oxidized states of a redox-active probe. Without intending to be limiting on the scope of embodiments herein, this example demonstrated that the excitation wavelength and the effective refractive index nsin(θ) are the main parameters affecting ΔI under potential modulations. Moreover, interrogating the EC-SPR system 310 in the linear region of the resonant angle of the conventional SPR curve and tuning the excitation light to the absorption band of the redox probe lead to maximum ΔI under potential modulation and therefore enhanced the sensitivity of detection.

Although the descriptions herein related to EC-SPR have been discussed and illustrated in the context of an exemplary process, it will be appreciated that many other variations and embodiments are within the scope of these teachings, and the focus on the exemplary embodiment is not intended to be limiting. With such improvements as described, it is anticipated that an EC-SPR system will provide improved detection abilities related to sensitivity in the study of surface-confined redox species at low surface densities is increased and overcomes the effects of background signals in biosensor applications. Furthermore, the improvements according to multiple embodiments and alternatives, are expected to be useful at wavelengths shorter than 630 nm and thus should permit the use of at least the following redox couples nile red, nile blue, viologen, and anthraquinone. The EC-SPR system within present embodiments was also easier to fabricate because it did not require the use of any grating couplers.

Accordingly, it will be understood that the embodiments described herein are not limited in their application to the details of the teachings and descriptions set forth, or as illustrated in the accompanying figures. Rather, it will be understood that the present embodiments and alternatives, as described and claimed herein, are capable of being practiced or carried out in various ways. Also, it is to be understood that words and phrases used herein are for the purpose of description and should not be regarded as limiting. The use herein of such words and phrases as “such as,” “comprising,” “e.g.,” “containing,” or “having” and variations of those words is meant to encompass the items listed thereafter, and equivalents of those, as well as additional items. The use of “including” (or, “include,” etc.) should be interpreted as “including but not limited to.”

Accordingly, the foregoing descriptions of several embodiments and alternatives are meant to illustrate, rather than to serve as limits on the scope of what has been disclosed herein. It will be understood by those having ordinary skill in the art that modifications and variations of these embodiments are reasonably possible in light of the above teachings and descriptions.

What is claimed is:

1. A spectroelectrochemical detection system, comprising:
a light source that generates light waves; and
a coated substrate for receiving an electroactive probe, wherein the coated substrate comprises a first layer which is a silver (Ag) layer and a second layer which is a gold (Au) layer to form a bilayer, and wherein the gold (Au) layer is arranged to isolate the silver (Ag) layer from an operating environment.

2. The spectroelectrochemical device of claim 1, wherein a thickness of the silver (Ag) layer is between 10-60 nm and a thickness of the gold (Au) layer is between 2-50 nm.

3. The spectroelectrochemical detection system of claim 1, wherein the coated substrate further comprises a third layer configured to receive the electroactive probe.

4. The spectroelectrochemical detection system of claim 3, wherein the third layer comprises a 3-Mercaptopropionic acid (MPA) surface coating.

5. The spectroelectrochemical detection system of claim 1, wherein the electroactive probe comprises a protein and an electroactive species that undergoes reduction and oxidation in response to an applied electric potential and impacts propagation of the light waves.

6. The spectroelectrochemical detection system of claim 5, wherein the electroactive species is selected from the group consisting of cytochrome c, nile red, nile blue, viologen, and anthraquinone.

7. The spectroelectrochemical detection system of claim 1, wherein the light source is a continuous wave laser.

8. The spectroelectrochemical detection system of claim 7, wherein the continuous wave laser is a surface plasmon wave device.

9. The spectroelectrochemical detection system of claim 1, wherein a thickness of the silver (Ag) layer is 35 nm and a thickness of the gold (Au) layer is 5 nm.
